(19) United States
(12) Patent Application Publication
(10) Pub. No.: US 2008/0226577 A1
(43) Pub. Date: Sep. 18, 2008

(54) Cosmetic product comprising silicone compounds and an amphiphilic polymer

(75) Inventors: Florence L'Alloret, Paris (FR); Danielle Millecamps, Vigneux Sur Seine (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/003,023

(22) Filed: Dec. 19, 2007

Related U.S. Application Data
(60) Provisional application No. 60/883,169, filed on Jan. 3, 2007.

(30) Foreign Application Priority Data
Dec. 20, 2006 (FR) 06 55682

(51) Int. Cl. A61K 8/81; A61K 8/89; A61Q 5/00

(52) U.S. Cl. 424/70.12; 424/70.11

(57) ABSTRACT

The present invention relates to a cosmetic kit that can be used for the care and/or make-up of keratinous substance(s), comprising at least two compositions that are different and are packaged separately with at least one of the compositions being of the emulsion type and comprising at least one amphiphilic polymer, the kit comprising one or more compounds X, one or more compounds Y, and at least one catalyst, with at least one of compounds X and Y being a silicone compound and said compounds X and Y being capable of reacting together by a hydrosilylation reaction, when they are brought into contact with one another, in the presence of a catalyst, and in which the compounds X, Y and the catalyst, are not present simultaneously in the same composition.

This non provisional application claims the benefit of French Application No. 06 55682 filed on Dec. 20, 2006 and U.S. Provisional Application No. 60/883,169 filed on Jan. 3, 2207.

The present invention mainly relates to a cosmetic kit, notably for make-up and/or care of keratinous substances, comprising a first composition containing at least one compound X and a second composition containing at least one compound Y, compounds X and Y being capable of reacting together if necessary in the presence of a catalyst or of a peroxide, and at least one of the compounds being a silicone compound with at least one of the compositions additionally containing an amphiphilic polymer.

The kit according to the invention can be a product for make-up and/or care of keratinous substances including keratin fibres, in particular the skin, the lips, the eyelashes, the eyebrows or the nails.

In the field of make-up and/or cosmetic care, a large number of compositions are in the form of emulsions. These emulsions generally contain one or more emulsifier(s) selected from the amphoteric, anionic, cationic or nonionic emulsifiers, used alone or mixed, and optionally a co-emulsifier.

The emulsifiers are selected appropriately depending on the emulsion to be produced (W/O or O/W).

Among the emulsifiers that can be used, we may mention more particularly the amphiphilic polymers, which are greatly valued in the field of cosmetics owing to the performance with which they are associated.

Thus, emulsions stabilized with amphiphilic polymers are particularly stable and lead to novel textural properties relative to emulsions stabilized with surfactants of low molar mass. These emulsions can moreover comprise fatty phases of very varied natures: triglycerides, alkanes, esters, silicones, sun filters, perfluorinated compounds, either alone or mixed. Finally, the ratio of the concentration of amphiphilic polymers to the proportion of dispersed phase is low (about 0.1) relative to the case of emulsions stabilized with surfactants (about 0.4).

However, after application on the skin, the fatty phase of these emulsions can lead to a relatively shiny skin, this effect being amplified with the proportion of oil and with the presence of non-volatile oil.

Now, although these properties of gloss are required for certain cosmetic products such as lipsticks and nail varnishes, they may conversely be undesirable for other cosmetic products such as foundations and care products.

Consequently, the present invention aims more particularly to propose cosmetic products for make-up and/or care incorporating amphiphilic polymers but instead having a reduced gloss effect or even without any gloss effect.

Unexpectedly, the inventors discovered that it is possible to meet these requirements provided the cosmetic emulsions in question are formulated with a system of specific polymers.

This system brings together two compounds, notably silicone compounds, which when in contact, if necessary in the presence of a catalyst or of a peroxide, polymerize in situ at atmospheric pressure and at room temperature, to form films that are advantageously biocompatible and non-sticky. Such systems are notably described partly in documents WO 01/96450 and GB 2 407 496.

These polymeric films, which can be formed in situ on a substrate, notably such as keratinous material, turn out to have advantageous properties in cosmetic terms, namely good adhesion, good durability and comfort.

Thus, according to a first aspect, the present invention relates to a cosmetic kit that can be used for the care and/or make-up of keratinous substance(s), notably of the skin, comprising at least two compositions that are different and are packaged separately with at least one of the compositions being of the emulsion type and comprising at least one amphiphilic polymer, the kit comprising one or more compounds X, one or more compounds Y, and optionally at least one catalyst or a peroxide, with at least one of compounds X and Y being a silicone compound and said compounds X and Y being capable of reacting together by a hydrosilylation reaction in the presence of a catalyst, or by a condensation reaction, or by a crosslinking reaction in the presence of a peroxide, when they are brought into contact with one another, if necessary in the presence of a catalyst or of a peroxide, and in which the compounds X, Y and the catalyst or the peroxide, when it is present, are not present simultaneously in the same composition.

According to a particular embodiment, the kit comprises at least:

i. a first composition containing, in a physiologically acceptable medium, at least one compound X and

ii. a second composition containing, in a physiologically acceptable medium, at least one compound Y,

with at least one of said first and second compositions being in the form of an emulsion and containing at least one amphiphilic polymer, and at least one of said first and second compositions additionally containing if necessary at least one catalyst or a peroxide.

According to an alternative embodiment, the first and second compositions are both emulsions stabilized respectively with at least one amphiphilic polymer.

During mixing of the cosmetic compositions containing them, compounds X and Y polymerize in situ, adhere to the keratinous substances and make it possible to obtain less glossy deposits on the skin.

Preferably, the first composition that includes compound X and the second composition that includes compound Y are packaged in separate packaging.

For example, each composition can be packaged separately in the same packaging article, for example in a two-compartment pen, the base composition being delivered by one end of the pen and the top composition being delivered by the other end of the pen, each end being closed notably hermetically with a cap. Each composition can also be packaged in a compartment within the same packaging article, the two compositions being mixed at the end or ends of the packaging article during delivery of each composition.

Alternatively, each of the first and second compositions can be packaged in a different packaging article.

The invention also relates to a method of cosmetic care and/or make-up of keratinous substance(s) comprising at least the application on keratinous substances (a) of at least one amphiphilic polymer, (b) of one or more compounds X, (c) of one or more compounds Y, with at least one of compounds X and Y being a silicone compound and said compounds X and Y being capable of reacting together by a hydrosilylation reaction in the presence of a catalyst, or by a condensation reaction, or by a crosslinking reaction in the presence of a peroxide, when they are brought into contact with one another, and (d) if necessary of at least one catalyst or a peroxide, and applications (a), (b), (c) and (d) can be simultaneous or consecutive in any order provided that it promotes the interaction of said compounds X and Y.

Thus, the compound or compounds X, the compound or compounds Y, can be applied on keratinous substances from several compositions, at least one of which is in the form of an emulsion, the compositions containing respectively the compound or compounds X, the compound or compounds Y, the amphiphilic polymer or polymers, on their own or mixed, or from a single composition containing the compound or compounds X, the compound or compounds Y, and the amphiphilic polymer or polymers.

According to a particular embodiment of the invention, a first composition containing at least the compound or compounds X, and a second composition containing at least the compound or compounds Y, are applied on the keratin fibres, with at least one of said first and second compositions being in the form of an emulsion and additionally containing at least one amphiphilic polymer, at least one of the first and second compositions additionally containing if necessary at least one catalyst or a peroxide.

More particularly, said method can comprise applying, on said keratinous substances, at least one composition of the emulsion type containing, in a physiologically acceptable medium, at least one compound X, at least one compound Y, if applicable, at least one catalyst or a peroxide and at least one amphiphilic polymer.

The invention also relates to a cosmetic method for make-up and/or care of keratinous substances, notably of human skin, comprising the application on said keratinous substances:
of at least one layer of a first composition containing, in a physiologically acceptable medium, at least one compound X;
of at least one layer of a second composition containing, in a physiologically acceptable medium, at least one compound Y,

at least one of compounds X and Y being a silicone compound, said compounds X and Y being capable of reacting together by a hydrosilylation reaction in the presence of a catalyst, or by a condensation reaction, or by a crosslinking reaction in the presence of a peroxide, when they are brought into contact with one another, with at least one of said first and second compositions being an emulsion and comprising at least one amphiphilic polymer, and

at least one of the first and second compositions additionally containing if necessary at least one catalyst or a peroxide.

According to a variant, the method comprises applying, on keratinous substances, at least one layer of the second composition that includes compound Y and if necessary an amphiphilic polymer, then depositing on the layer or layers of said second composition, at least one layer of the first composition that includes compound X and if necessary an amphiphilic polymer, at least one of the first and second compositions additionally containing if necessary at least one catalyst or a peroxide.

According to another variant of this method, it is possible to apply, on the keratinous substances, at least one layer of the first composition that includes compound X and if necessary at least one amphiphilic polymer, and then deposit on the layer or layers of said first composition, at least one layer of the second composition that includes compound Y and if necessary at least one amphiphilic polymer, at least one of the first and second compositions additionally containing if necessary at least one catalyst or a peroxide.

Several layers of each of the first and second compositions can also be applied alternately on keratinous substances.

The composition applied can also be obtained by mixing, at the time of use, a first composition containing at least compound X and a second composition containing at least compound Y, at least one of the first and second compositions being an emulsion and comprising at least one amphiphilic polymer, and at least one of the first and second compositions additionally containing if necessary at least one catalyst or a peroxide.

In the sense of the invention, notably in the embodiment where the composition is obtained as described above, namely by mixing, at the time of use, a first composition containing at least compound X and a second composition containing at least compound Y, it is to be understood that the mixture thus formed comprises compounds X and/or Y′ in a form that has not yet reacted and not exclusively in the form of their reaction product by hydrosilylation, by polycondensation and/or by crosslinking in the presence of a peroxide.

Thus, formation of the reaction product according to the invention can either be carried out directly on the surface of the keratinous substance that is to be treated, or initiated just before application by extemporaneous mixing of compounds X and Y in conditions favourable for their interaction, formation of the reaction product being in the latter case finalized on the surface of the keratinous substance.

For obvious reasons, and in view of the great reactivity of compounds X and/or Y, it is in fact necessary that their application should be carried out in conditions that are favourable for the manageability of the composition containing it (or them) notably with respect to its spreading, for example. The method according to the invention therefore employs a composition containing compounds X and Y, and therefore not congealed in the form of the expected final film resulting from reaction of all of X and/or of all of Y.

According to one embodiment, the method includes a supplementary stage comprising depositing, on the layer or layers of compositions comprising X and Y, at least one layer of a third composition comprising a film-forming polymer and an organic or aqueous solvent medium.

According to another variant, the composition applied contains at least one of compounds X and Y in an encapsulated form.

Thus, the present invention also relates to a cosmetic composition, notably for care and/or make-up of keratinous substances, of the emulsion type, containing, in a physiologically acceptable medium, at least one compound X, a compound Y and if applicable at least the catalyst and the peroxide if necessary for interaction of said compounds X and Y and at least one amphiphilic polymer as defined hereunder with at least one of compounds X and Y being in an encapsulated form. According to a preferred variant of this embodiment, the two compounds X and Y are present in separate encapsulated forms.

According to this embodiment, the two compounds X and Y can be packaged in one and the same composition while avoiding the risk of premature reaction between them. Said reaction only occurs at the moment when the composition is manipulated prior to or at the moment of its application on the keratinous substance. The encapsulated form or forms of compound X and/or Y break on drying and compounds X and Y, which then come into contact, react to form the expected film.

Compounds X and Y

Silicone compound means a polyorganosiloxane compound, i.e. comprising at least two organosiloxane units, for example at least 5 organosiloxane units, notably at least 10 organosiloxane units. According to a particular embodiment, at least one of compounds X and Y, or compounds X and compounds Y are silicone compounds. Compounds X and Y can be aminated or non-aminated.

According to another embodiment, at least one of compounds X and Y is a polymer whose main chain is formed primarily of organosiloxane units. Among the silicone compounds mentioned below, some may display both film-forming and adhesive properties, depending for example on their proportion of silicone or depending on whether they are used mixed with a particular additive. It is therefore possible to adjust the film-forming properties or the adhesive properties of said compounds according to the proposed use, which is the case in particular for the so-called “room temperature vulcanization” reactive elastomeric silicones.

Compounds X and Y can react with each other at a temperature varying between room temperature and 180° C. Advantageously, compounds X and Y are capable of reacting together at room temperature (20±5° C.) and atmospheric pressure, or advantageously in the presence of a catalyst, by a hydrosilylation reaction or a condensation reaction, or a crosslinking reaction in the presence of a peroxide.

Polar Groups

According to a particular embodiment, at least one of compounds X and Y, for example compound X, bears at least one polar group that is able to form at least one hydrogen bond with keratinous substances.

By polar group, we mean a group having carbon atoms and hydrogen atoms in its chemical structure and at least one heteroatom (such as O, N, S and P), such that said group is able to establish at least one hydrogen bond with keratinous substances.

Compounds bearing at least one group that can form a hydrogen bond are particularly advantageous, as they endow the compositions containing them with better adherence on keratinous substances.

The polar group or groups borne by at least one of compounds X and Y is/are able to establish a hydrogen bond, and include either a hydrogen atom bound to an electronegative atom, or an electronegative atom for example an oxygen, nitrogen or sulphur atom. When the group has a hydrogen atom bound to an electronegative atom, the hydrogen atom can interact with another electronegative atom borne for example by another molecule, such as keratin, to form a hydrogen bond. When the group has an electronegative atom, the electronegative atom can interact with a hydrogen atom bound to an electronegative atom borne for example by another molecule, such as keratin, to form a hydrogen bond.

Advantageously, these polar groups can be selected from the following groups:
carboxylic acids —COOH,
alcohols, such as: —CH₂OH or —CH(R)OH, R being an alkyl radical having from 1 to 6 carbon atoms,
amino of formula —NR₁R₂, in which R₁ and R₂, which may be identical or different, represent an alkyl radical having from 1 to 6 carbon atoms or one of R₁ or R₂ denotes a hydrogen atom, and the other one of R₁ and R₂ represents an alkyl radical having from 1 to 6 carbon atoms,
pyridino,
amido of formula —NH—COR′ or —CO—NH—R′ in which R′ represents a hydrogen atom or an alkyl radical having from 1 to 6 carbon atoms,
pyrrolidino preferably selected from the groups of formula:

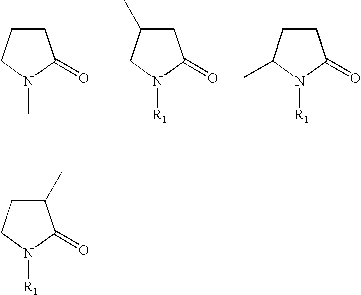

R₁ being an alkyl radical having from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulphonamido such as —NR′—S(═O)₂—R′, R′ corresponding to the above definition.

Preferably, these polar groups are present at a content less than or equal to 10 wt. % relative to the weight of each compound X or Y, preferably less than or equal to 5 wt. %, for example at a content ranging from 1 to 3 wt. %.

The polar group or groups can be located in the main chain of compound X and/or Y or can be pendant from the main chain or located at the ends of the main chain of compound X and/or Y.

1—Compounds X and Y Capable of Reacting by Hydrosilylation

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one amphiphilic polymer, the kit comprising one or more compounds X, one or more compounds Y, and at least one catalyst, with at least one of compounds X and Y being a silicone compound and said compounds X and Y being capable of reacting together by a hydrosilylation reaction when they are brought into contact with one another in the presence of a catalyst, and in which the compounds X, Y and the catalyst are not present simultaneously in the same composition.

According to this embodiment, compounds X and Y are capable of reacting by hydrosilylation in the presence of a catalyst, said reaction being represented schematically in a simplified manner as follows:

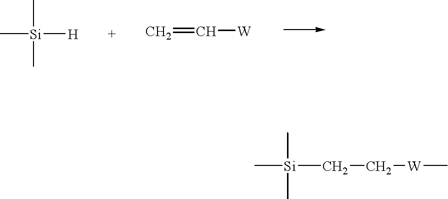

with W representing a carbon chain and/or silicone chain containing one or more unsaturated aliphatic groups.

In this case, compound X can be selected from silicone compounds comprising at least two unsaturated aliphatic groups. As an example, compound X can be a polyorganosiloxane comprising a silicone main chain whose unsaturated aliphatic groups are pendant from the main chain (side group) or located at the ends of the main chain of the compound (end group). These particular compounds will be called, hereinafter, polyorganosiloxanes with unsaturated aliphatic groups.

According to one embodiment, compound X and/or compound Y bear at least one polar group, as described above, capable of forming at least one hydrogen bond with keratinous substances. This polar group is advantageously carried by compound X, which has at least two unsaturated aliphatic groups.

According to one embodiment, compound X is selected from the polyorganosiloxanes comprising at least two unsaturated aliphatic groups, for example two or three vinyl or allyl groups, each attached to a silicon atom.

According to an advantageous embodiment, compound X is selected from the polyorganosiloxanes containing siloxane units of formula:

$${R}_{m} {R}^{′} {SiO}_{( \frac{3 - m}{2} )} \qquad ( I )$$

in which:
R represents a linear or cyclic, monovalent hydrocarbon group, having from 1 to 30 carbon atoms, preferably from 1 to 20, and better still from 1 to 10 carbon atoms, for example a short-chain alkyl radical, comprising for example from 1 to 10 carbon atoms, in particular a methyl radical or alternatively a phenyl group, preferably a methyl radical,
m is equal to 1 or 2 and
R′ represents:
   an unsaturated aliphatic hydrocarbon group having from 2 to 10, preferably from 3 to 5 carbon atoms, for example a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom; we may mention, as group R′, the vinyl and allyl groups and mixtures thereof; or
   an unsaturated cyclic hydrocarbon group having from 5 to 8 carbon atoms, for example a cyclohexenyl group.

Preferably R′ is an unsaturated aliphatic hydrocarbon group, preferably a vinyl group.

According to one embodiment, R represents an alkyl radical having from 1 to 10 carbon atoms or alternatively a phenyl group, and preferably a methyl radical, and R′ is a vinyl group.

According to a particular embodiment, the polyorganosiloxane also contains units of formula:

$${R}_{n} {SiO}_{( \frac{4 - n}{2} )} \qquad ( II )$$

in which R is a group as defined previously, and n is equal to 1, 2 or 3.

According to a variant, compound X can be a silicone resin comprising at least two ethylenic unsaturations, said resin being capable of reacting with compound Y by hydrosilylation in the presence of a catalyst. We may mention for example the resins of type MQ or MT which themselves bear —CH═CH₂ unsaturated reactive end groups.

These resins are crosslinked organosiloxane polymers.

The class of the silicone resins is known by the name “MDTQ”, the resin being described in relation to the different siloxane monomer units that it contains, each of the letters “MDTQ” characterizing a type of unit.

The letter M represents the monofunctional unit of formula (CH₃)₃SiO_{1/2}, the silicon atom being bound to a single oxygen atom in the polymer comprising said unit.

The letter D denotes a bifunctional unit (CH₃)₂SiO₂/2 in which the silicon atom is bound to two oxygen atoms.

The letter T represents a trifunctional unit of formula (CH₃)SiO_{3/2}.

In units M, D, T defined above, at least one of the methyl groups can be substituted with a group R other than the methyl group, such as a hydrocarbon radical (notably alkyl) having from 2 to 10 carbon atoms or a phenyl group or alternatively a hydroxyl group.

Finally, the letter Q denotes a tetrafunctional unit SiO_{4/2} in which the silicon atom is bound to four hydrogen atoms which are themselves attached to the rest of the polymer. As examples of said resins, we may mention the MT silicone resins such as poly(phenyl-vinylsilsesquioxane) such as that marketed under the reference SST-3PV1 by the company Gelest.

Preferably, compounds X have from 0.01 to 1 wt. % of unsaturated aliphatic groups.

Advantageously, compound X is selected from the polyorganopolysiloxanes, notably those comprising the siloxane units (I) and optionally (II) described previously.

Compound Y preferably has at least two free Si—H groups (hydrogenosilane groups).

Compound Y can be selected advantageously from the polyorganosiloxanes comprising at least one alkylhydrogenosiloxane unit of the following formula:

$${R}_{p} H {SiO}_{( \frac{3 - p}{2} )} \qquad ( III )$$

in which:

R represents a linear or cyclic, monovalent hydrocarbon group, having from 1 to 30 carbon atoms, for example an alkyl radical having from 1 to 30 carbon atoms, preferably from 1 to 20 and better still from 1 to 10 carbon atoms, in particular a methyl radical, or alternatively a phenyl group and p is equal to 1 or 2. Preferably R is a hydrocarbon group, preferably methyl.

These polyorganosiloxane compounds Y with alkylhydrogenosiloxane units can additionally contain units of formula:

$${R}_{n} {SiO}_{( \frac{4 - n}{2} )} \qquad ( II )$$

as defined above.

Compound Y can be a silicone resin comprising at least one unit selected from the units M, D, T, Q as defined above and comprising at least one Si—H group such as the poly(methyl-hydridosilsesquioxane) marketed under the reference SST-3 MH1.1 by the company Gelest.

Preferably, these polyorganosiloxane compounds Y have from 0.5 to 2.5 wt. % of Si—H groups.

Advantageously, the radicals R represent a methyl group in formulae (I), (II), (III) above.

Preferably, these polyorganosiloxanes Y have end groups of formula (CH₃)₃SiO_{1/2}.

Advantageously, the polyorganosiloxanes Y have at least two alkylhydrogenosiloxane units of formula —(H₃C)(H)SiO— and optionally include —(H₃C)₂SiO— units.

These polyorganosiloxane compounds Y with hydrogenosilane groups are described for example in document EP 0465744.

According to one variant, compound X is selected from the organic oligomers or polymers (by organic, we mean compounds whose main chain is not a silicone chain, preferably compounds not containing silicon atoms) or from hybrid organic/silicone polymers or oligomers, said oligomers or polymers bearing at least 2 unsaturated reactive aliphatic groups, compound Y being selected from the polyorganosiloxanes Y with hydrogenosilane groups mentioned above.

According to one embodiment, the organic or hybrid organic/silicone compounds X bearing at least 2 unsaturated reactive aliphatic groups, have at least one polar group as described above.

Compound X, of organic nature, can then be selected from the vinylic, (meth)acrylic polymers or oligomers, polyesters, polyurethanes and/or polyureas, polyethers, perfluoropolyethers, polyolefins such as polybutene, polyisobutylene, dendrimers or organic hyperbranched polymers, or mixtures thereof.

In particular, the organic polymer or the organic moiety of the hybrid polymer can be selected from the following polymers:

a) polyesters with ethylenic unsaturation(s):

This is a group of polymers of the polyester type having at least 2 ethylenic double bonds, randomly distributed in the main chain of the polymer. These unsaturated polyesters are obtained by polycondensation of a mixture:
of linear or branched aliphatic or cycloaliphatic dicarboxylic acids notably having 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with ethylenic unsaturations such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and/or of diol dimers resulting from reduction of dimers of fatty acids as defined previously, and
of one or more dicarboxylic acids or their anhydrides having at least one polymerizable ethylenic double bond and having from 3 to 50 carbon atoms, preferably from 3 to 20 and better still from 3 to 10 carbon atoms, such as maleic acid, fumaric acid or itaconic acid.

b) polyesters with (meth)acrylate side and/or end groups:

This is a group of polymers of the polyester type obtained by polycondensation of a mixture:
of linear or branched aliphatic or cycloaliphatic dicarboxylic acids notably having from 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with an ethylenic unsaturation such as the dimers of oleic or linoleic acids described in application EP-A-959 066 (paragraph [0021]) marketed under the designations Pripol® by the company Unichema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl(meth)acrylate and glycerol methacrylate.

These polyesters differ from those described above in section a) by the fact that the ethylenic double bonds are not located in the main chain but on side groups or at the end of the chains. These ethylenic double bonds are those of the (meth)acrylate groups present in the polymer.

Such polyesters are marketed for example by the company UCB under the designations EBECRYL® (EBECRYL® 450: molecular weight 1600, on average 6 acrylate functions per molecule, EBECRYL® 652: molecular weight 1500, on average 6 acrylate functions per molecule, EBECRYL® 800: molecular weight 780, on average 4 acrylate functions per molecule, EBECRYL® 810: molecular weight 1000, on average 4 acrylate functions per molecule, EBECRYL® 50 000: molecular weight 1500, on average 6 acrylate functions per molecule).

c) polyurethanes and/or polyureas with (meth)acrylate groups, obtained by polycondensation:
of aliphatic, cycloaliphatic and/or aromatic diisocyanates, triisocyanates and/or polyisocyanates notably having from 4 to 50, preferably from 4 to 30 carbon atoms, such as hexamethylenediisocyanate, isophoronediisocyanate, toluenediisocyanate, diphenylmethanediisocyanate or isocyanurates of formula:

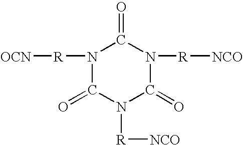

resulting from the trimerization of 3 molecules of diisocyanates OCN—R—CNO,
where R is a linear, branched or cyclic hydrocarbon radical having from 2 to 30 carbon atoms;
of polyols, notably of diols, free from polymerizable ethylenic unsaturations, such as 1,4-butanediol, ethylene glycol or trimethylolpropane, and/or of polyamines, notably of aliphatic, cycloaliphatic and/or aromatic diamines, notably having from 3 to 50 carbon atoms, such as ethylenediamine or hexamethylenediamine, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl(meth)acrylate and glycerol methacrylate.

These polyurethanes/polyureas with acrylate groups are marketed for example under the designation SR 368 (tris(2-hydroxyethyl)isocyanurate-triacrylate) or CRAYNOR® 435 by the company CRAY VALLEY, or under the designation EBECRYL® by the company UCB (EBECRYL® 210: molecular weight 1500, 2 acrylate functions per molecule, EBECRYL® 230: molecular weight 5000, 2 acrylate functions per molecule, EBECRYL® 270: molecular weight 1500, 2 acrylate functions per molecule, EBECRYL® 8402: molecular weight 1000, 2 acrylate functions per molecule, EBECRYL® 8804: molecular weight 1300, 2 acrylate functions per molecule, EBECRYL® 220: molecular weight 1000, 6 acrylate functions per molecule, EBECRYL® 2220: molecular weight 1200, 6 acrylate functions per molecule, EBECRYL® 1290: molecular weight 1000, 6 acrylate functions per molecule, EBECRYL® 800: molecular weight 800, 6 acrylate functions per molecule).

We may also mention the water-soluble aliphatic diacrylate polyurethanes marketed under the designations EBECRYL® 2000, EBECRYL® 2001 and EBECRYL® 2002, and the diacrylate polyurethanes in aqueous dispersion marketed under the trade names IRR® 390, IRR® 400, IRR® 422 IRR® 424 by the company UCB.

d) polyethers with (meth)acrylate groups obtained by esterification, by (meth)acrylic acid, of the hydroxyl end groups of homopolymers or of C₁₋₄ alkylene glycol copolymers, such as polyethylene glycol, polypropylene glycol, copolymers of ethylene oxide and of propylene oxide preferably having a weight-average molecular weight below 10 000, polyethoxylated or polypropoxylated trimethylolpropane.

Di(meth)acrylate polyoxyethylenes of suitable molecular weight are marketed for example under the designations SR 259, SR 344, SR 610, SR 210, SR 603 and SR 252 by the company CRAY VALLEY or under the designation EBECRYL® 11 by UCB. Polyethoxylated trimethylolpropane triacrylates are marketed for example under the designations SR 454, SR 498, SR 502, SR 9035, SR 415 by the company CRAY VALLEY or under the designation EBECRYL® 160 by the company UCB. Polypropoxylated trimethylolpropane triacrylates are marketed for example under the designations SR 492 and SR 501 by the company CRAY VALLEY.

e) epoxyacrylates obtained by reaction between
at least one diepoxide selected for example from:
   (i) bisphenol A diglycidyl ether,
   (ii) a diepoxy resin resulting from the reaction between bisphenol A diglycidyl ether and epichlorohydrin,
   (iii) an epoxyester resin with α,ω-diepoxy end groups resulting from the condensation of a dicarboxylic acid having from 3 to 50 carbon atoms with a stoichiometric excess of (i) and/or (ii),
   (iv) an epoxyether resin with α,ω-diepoxy end groups resulting from the condensation of a diol having from 3 to 50 carbon atoms with a stoichiometric excess of (i) and/or (ii),
   (v) natural or synthetic oils bearing at least 2 epoxide groups, such as epoxidized soya oil, epoxidized linseed oil and epoxidized vernonia oil,
   (vi) a phenol-formaldehyde polycondensate (Novolac® resin), of which the end groups and/or side groups have been epoxidized,

and
one or more carboxylic acids or carboxylic polyacids having at least one ethylenic double bond at α,β of the carboxyl group such as (meth)acrylic acid or crotonic acid or esters of (meth)acrylic acid and of a diol or polyol having from 2 to 20 carbon atoms, preferably from 2 to 6 carbon atoms such as 2-hydroxyethyl(meth)acrylate.

Such polymers are marketed for example under the designations SR 349, SR 601, CD 541, SR 602, SR 9036, SR 348, CD 540, SR 480, CD 9038 by the company CRAY VALLEY, under the designations EBECRYL® 600 and EBECRYL® 609, EBECRYL® 150, EBECRYL® 860, EBECRYL® 3702 by the company UCB and under the designations PHOTOMER® 3005 and PHOTOMER® 3082 by the company HENKEL.

f) (C₁₋₅₀ alkyl) poly(meth)acrylates, said alkyl being linear, branched or cyclic, bearing at least two functions with ethylenic double bond carried by the lateral and/or terminal hydrocarbon chains.

Such copolymers are marketed for example under the designations IRR® 375, OTAS 480 and EBECRYL® 2047 by the company UCB.

g) polyolefins such as polybutene, polyisobutylene,

h) perfluoropolyethers with acrylate groups obtained by esterification, for example by (meth)acrylic acid, of perfluoropolyethers bearing hydroxyl side and/or end groups.

These α,ω-diol perfluoropolyethers are described notably in EP-A-1057849 and are marketed by the company AUSIMONT under the designation FOMBLIN® Z DIOL.

i) dendrimers and hyperbranched polymers bearing (meth)acrylate or (meth)acrylamide end groups obtained respectively by esterification or amidation of dendrimers and of hyperbranched polymers with hydroxyl or amino terminal functions, by (meth)acrylic acid.

The dendrimers (from the Greek dendron=tree) are “tree-like” polymer molecules, i.e. highly branched, invented by D. A. Tomalia and his team at the beginning of the 1990's (Donald A. Tomalia et al., Angewandte Chemie, Int. Engl. Ed., Vol. 29, No. 2, pages 138-175). They are structures constructed around a, generally polyvalent, central unit. Branched chain-extending units are arranged according to a perfectly defined structure around this central unit, thus giving rise to symmetrical, monodispersed macromolecules having a well-defined chemical and stereochemical structure. Dendrimers of the polyamidoamine type are marketed for example under the name STARBURST® by the company DENDRITECH.

The hyperbranched polymers are polycondensates, generally of the polyester, polyamide or polyethyleneamine type, obtained from multifunctional monomers, which have a tree-like structure similar to that of the dendrimers but far less regular than the latter (see for example WO-A-93/17060 and WO 96/12754).

The company PERSTORP markets hyperbranched polyesters under the name BOLTORN®. Hyperbranched polyethyleneamines are available under the name COMBURST® from the company DENDRITECH. Hyperbranched poly(esteramide)s with hydroxyl end groups are marketed by the company DSM under the name HYBRANE®.

These dendrimers and hyperbranched polymers, esterified or amidated by acrylic and/or methacrylic acid, differ from the polymers described in sections a) to h) above by the very large number of ethylenic double bonds present. This increased functionality, generally greater than 5, makes them particularly useful in enabling them to act as a “crosslinking node”, i.e. a multiple crosslinking site.

It is therefore possible to use these dendritic and hyperbranched polymers in association with one or more of the above polymers and/or oligomers a) to h).

1a—Additional Reactive Compounds

According to one embodiment, the compositions containing compound X and/or Y can additionally comprise an additional reactive compound such as:
organic or mineral particles having on their surface at least 2 unsaturated aliphatic groups—we may mention for example the silicas surface-treated for example with silicone compounds with vinylic groups such as for example cyclotetramethyltetravinylsiloxane-treated silica,
silazane compounds such as hexamethyldisilazane.

1b—Catalyst

The hydrosilylation reaction takes place in the presence of a catalyst which can be present with one or other of the compounds X or Y or can be present on its own. For example, this catalyst can be present in the composition in an encapsulated form if the two compounds X and Y, which it must cause to interact, are present in this same composition in an unencapsulated form or conversely it can be contained there in an unencapsulated form if at least one of compounds X and Y is present in the composition in an encapsulated form. The catalyst is preferably based on platinum or tin.

We may mention for example platinum-based catalysts deposited on a support of silica gel or of powdered charcoal, platinum chloride, salts of platinum and of chloroplatinic acids.

The chloroplatinic acids are preferably used in hexahydrate or anhydrous form, which are easily dispersible in organosilicone media.

We may also mention platinum complexes, such as those based on chloroplatinic acid hexahydrate and divinyl tetramethyldisiloxane.

The catalyst can be present at a content in the range from 0.0001 to 20 wt. % relative to the total weight of the composition containing it.

Compounds X and/or Y can be combined with polymerization inhibitors or retarders, and more particularly inhibitors of the catalyst. Non-limitatively, we may mention cyclic polymethylvinylsiloxanes, and in particular tetravinyl tetramethyl cyclotetrasiloxane, acetylenic alcohols, preferably volatile, such as methylisobutynol.

The presence of ionic salts, such as sodium acetate, can have an influence on the rate of polymerization of the compounds.

As an example of a combination of compounds X and Y reacting by hydrosilylation in the presence of a catalyst, we may mention the following references offered by the company Dow Corning: DC7-9800 Soft Skin Adhesive Parts A & B, as well as the combination of the following mixtures A and B prepared by Dow Corning:

MIXTURE A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

Advantageously, compounds X and Y are selected from silicone compounds capable of reacting by hydrosilylation in the presence of a catalyst; in particular compound X is selected from the polyorganosiloxanes comprising units of formula (I) described above and compound Y is selected from organosiloxanes comprising alkylhydrogenosiloxane units of formula (III) described above.

According to a particular embodiment, compound X is a polydimethylsiloxane with vinylic end groups, and compound Y is a polymethylhydrogenosiloxane.

2/Compounds X and Y Capable of Reacting by Condensation

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one amphiphilic polymer, the kit comprising one or more compounds X, one or more compounds Y, and optionally at least one catalyst, with at least one of compounds X and Y being a silicone compound and said compounds X and Y being capable of reacting together by a condensation reaction when they are brought into contact with one another, and in which the compounds X, Y and the catalyst, when it is present, are not present simultaneously in the same composition.

According to this embodiment, compounds X and Y are capable of reacting by condensation, either in the presence of water (hydrolysis) by reaction of 2 compounds bearing alkoxysilane groups, or by so-called “direct” condensation by reaction of a compound bearing alkoxysilane group(s) and a compound bearing silanol group(s) or by reaction of 2 compounds bearing silanol group(s).

When the condensation is carried out in the presence of water, the latter can in particular be the ambient humidity, the residual water of the skin, of the lips, of the eyelashes and/or of the nails, or water from an external source, for example by prior moistening of the keratinous substance (for example by an atomizer, by natural or artificial tears).

In this manner of reaction by condensation, compounds X and Y, which may be identical or different, can therefore be selected from silicone compounds whose main chain contains at least two alkoxysilane groups and/or at least two silanol (Si—OH) side groups or end groups.

According to one embodiment, compound X and/or compound Y bears at least one polar group, as described above, capable of forming at least one hydrogen bond with keratinous substances.

According to an advantageous embodiment, compounds X and/or Y are selected from the polyorganosiloxanes comprising at least two alkoxysilane groups. By “alkoxysilane group”, we mean a group comprising at least one —Si—OR moiety, R being an alkyl group having from 1 to 6 carbon atoms.

Compounds X and Y are notably selected from the polyorganosiloxanes comprising alkoxysilane end groups, more specifically those which have at least 2 alkoxysilane end groups, preferably trialkoxysilane end groups.

These compounds X and/or Y preferably mostly comprise units of formula:

R⁹ₛSiO_{(4-s)/2}, (IV)

in which the groups R⁹ represent, independently of one another, a radical selected from alkyl groups having from 1 to 6 carbon atoms, phenyl groups, fluoroalkyl groups, and s is equal to 0, 1, 2 or 3. Preferably, groups R⁹ represent, independently of one another, an alkyl group having from 1 to 6 carbon atoms. As alkyl group, we may notably mention methyl, propyl, butyl, hexyl and mixtures thereof, preferably methyl or ethyl. As fluoroalkyl group, we may mention 3,3,3-trifluoropropyl.

According to a particular embodiment, compounds X and Y, which may be identical or different, are polyorganosiloxanes comprising units of formula:

(R⁹₂SiO₂)_{f}— (V)

in which R⁹ is as described above, preferably R⁹ is a methyl radical, and f is such that the polymer advantageously has a viscosity at 25° C. in the range from 0.5 to 3000 Pa.s, preferably in the range from 5 to 150 Pa.s; for example f can range from 2 to 5000, preferably from 3 to 3000, and more preferably from 5 to 1000.

These polyorganosiloxane compounds X and Y contain at least 2 trialkoxysilane end groups per molecule of polymer, said groups having the following formula

—ZSiR¹ₓ(OR)₃₋ₓ, (VI)

in which:

the radicals R represent, independently, a methyl, ethyl, n-propyl, isopropyl, n-butyl, sec-butyl, isobutyl group, preferably a methyl or ethyl group,

R¹ is a methyl or ethyl group,

x is equal to 0 or 1, preferably x is equal to 0 and

Z is selected from: the divalent hydrocarbon groups that do not have an ethylenic unsaturation and have from 1 to 18 carbon atoms, preferably from 2 to 18 carbon atoms (alkylene groups), the combinations of divalent hydrocarbon radicals and siloxane segments of the following formula (IX):

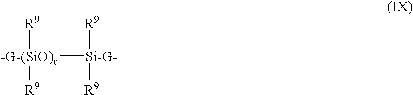

R⁹ being as described above, G is a divalent hydrocarbon radical without an ethylenic unsaturation and having from 1 to 18 carbon atoms, preferably from 2 to 18 carbon atoms and c is an integer in the range from 1 to 6.

Z and G can notably be selected from the alkylene groups such as methylene, ethylene, propylene, butylene, pentylene, hexylene, the arylene groups such as phenylene.

Preferably, Z is an alkylene group, and more preferably ethylene.

These polymers can have on average at least 1.2 trialkoxysilane end groups or terminal chains per molecule, and preferably on average at least 1.5 trialkoxysilane end groups per molecule. These polymers that can have at least 1.2 trialkoxysilane end groups per molecule, some can include other types of end groups such as end groups of formula CH₂═CH—SiR⁹₂— or of formula R⁶₃—Si—, in which R⁹ is as defined previously and each group R⁶ is selected independently from the R⁹ or vinyl groups. As examples of said end groups, we may mention the trimethoxysilane, triethoxysilane, vinyldimethoxysilane and vinylmethyloxyphenylsilane groups.

Such polymers are notably described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the contents of which are incorporated by reference in the present application.

We may mention, as compound X and/or Y, in particular the polyorganosiloxanes selected from the polymers of formula:

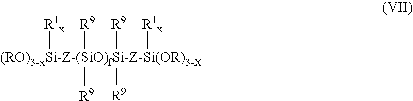

in which R, R¹, R⁹, Z, x and f are as described above.

Compounds X and/or Y can also include a mixture of polymers of formula (VII) above with polymers of the following formula (VIII):

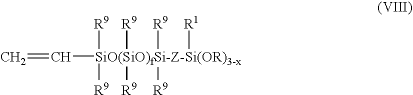

in which R, R¹, R⁹, Z, x, and f are as described above.

When the polyorganosiloxane compound X and/or Y with alkoxysilane group(s) includes said mixture, the various polyorganosiloxanes are present at contents such that the organosilyl terminal chains represent less than 40%, preferably less than 25% in number of terminal chains.

Polyorganosiloxane compounds X and/or Y that are particularly preferred are those of formula (VII) described above. Such compounds X and/or Y are described for example in document WO 01/96450.

As stated above, compounds X and Y can be identical or different.

In particular, compounds X and Y can represent a mixture of polydimethylsiloxanes with methoxysilane groups.

According to a variant, one of the 2 reacting compounds X or Y is of silicone character and the other is of organic character. For example, compound X is selected from organic oligomers or polymers or hybrid organic/silicone oligomers or polymers, said polymers or oligomers comprising at least two alkoxysilane groups, and Y is selected from silicone compounds such as the polyorganosiloxanes described above. In particular, the organic oligomers or polymers are selected from the vinylic, (meth)acrylic oligomers or polymers, polyesters, polyamides, polyurethanes and/or polyureas, polyethers, polyolefins, perfluoropolyethers, dendrimers and hyperbranched organic polymers, and mixtures thereof.

According to one embodiment, compound X of organic character or of hybrid organic/silicone character bears at least one polar group, as described above, capable of forming at least one hydrogen bond with the keratinous substance.

The organic polymers of vinylic or (meth)acrylic character, bearing alkoxysilane side groups, can in particular be obtained by copolymerization of at least one vinylic or (meth)acrylic organic monomer with a (meth)acryloxypropyltrimethoxysilane, a vinyltrimethoxysilane, a vinyltriethoxysilane, an allyltrimethoxysilane etc.

We may mention for example the (meth)acrylic polymers described in the document of KUSABE, M, Pitture e Verniei—European Coating; 12-B, pages 43-49, 2005, and notably the polyacrylates with alkoxysilane groups with the designation MAX from Kaneka or those described in the work by PROBSTER, M, Adhesion-Kleben & Dichten, 2004, 481 (1-2), pages 12-14.

The organic polymers resulting from a polycondensation or a polyaddition, such as polyesters, polyamides, polyurethanes and/or polyureas, polyethers, and bearing alkoxysilane side and/or end groups, can result for example from reaction of an oligomeric prepolymer as described above with one of the following silane reaction partners bearing at least one alkoxysilane group: aminopropyltrimethoxysilane, aminopropyltriethoxysilane, aminoethyl aminopropyl trimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes with alkoxysilane groups are described in the work by KUSABE, M., Pitture e Verniei—European Coating; 12-B, pages 43-49, 2005. As examples of polyurethanes with alkoxysilane end groups, we may mention those described in the document PROBSTER, M., Adhesion-Kleben & Dichten, 2004, 481 (1-2), pages 12-14 or alternatively those described in the document LANDON, S., Pitture e Verniei Vol. 73, No. 11, pages 18-24, 1997 or in the document HUANG, Mowo, Pitture e Verniei Vol. 5, 2000, pages 61-67, and we may notably mention the polyurethanes with alkoxysilane groups from OSI-WITCO-GE.

As polyorganosiloxane compounds X and/or Y, we may mention the resins of type MQ or MT which themselves bear alkoxysilane and/or silanol end groups, for example the poly(isobutylsilsesquioxane) resins functionalized with silanol groups offered under reference SST-S7C41 (three Si—OH groups) by the company Gelest.

2a—Additional Reactive Compound

According to one embodiment, compound X and/or Y can additionally be combined with an additional reactive compound comprising at least two alkoxysilane or silanol groups.

We may mention for example:
one or more organic or mineral particles with alkoxysilane and/or silanol groups on their surface, for example fillers surface-treated with said groups.

2b—Catalyst

The condensation reaction can take place in the presence of a metal-based catalyst which can be present with one or other of the compounds X or Y or can be present on its own. For example, said catalyst can be present in the composition in an encapsulated form if the two compounds X and Y, which it is to cause to interact, are present in this same composition in an unencapsulated form or conversely it can be present there in an unencapsulated form if at least one of compounds X and Y is present in the composition in an encapsulated form. The catalyst for use in this type of reaction is preferably a titanium-based catalyst.

We may notably mention the catalysts based on tetraalkoxytitanium of formula:

Ti(OR²)_{y}(OR³)_{4-y,}

in which R² is selected from the tertiary alkyl radicals such as tert-butyl, tert-amyl and 2,4-dimethyl-3-pentyl; R³ represents an alkyl radical having from 1 to 6 carbon atoms, preferably a methyl, ethyl, n-propyl, isopropyl, n-butyl, sec-butyl, hexyl group and y is a number in the range from 3 to 4, preferably from 3.4 to 4.

The catalyst can be present at a content ranging from 0.0001 to 20 wt. % relative to the total weight of the composition containing it.

2c—Diluent

The compositions that can be used, comprising X and/or Y, can additionally include a volatile silicone oil (or diluent) for lowering the viscosity of the composition. Said oil can be selected from the short-chain linear silicones such as hexamethyldisiloxane, octamethyltrisiloxane, cyclic silicones such as octamethylcyclotetrasiloxane, decamethylcyclopentasiloxane and mixtures thereof.

This silicone oil can represent from 5 to 95 wt. %, preferably from 10 to 80 wt. % relative to the weight of each composition.

As an example of a combination of compounds X and Y bearing alkoxysilane groups and reacting by condensation, we may mention the combination of the following mixtures A′ and B′ produced by the company Dow Corning:

Mixture A′:

| Ingredient (INCI name) | CAS No. | Contents (%) | Function |
|---|---|---|---|
| Bis- | PMN87176 | 25-45 | Polymer |
| Trimethoxysiloxyethyl | | | |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| Ingredient (INCI name) | CAS No. | Contents (%) | Function |
|---|---|---|---|
| Disiloxane | 107-46-0 | 80-99 | Solvent |
| Tetra T Butyl Titanate | — | 1-20 | Catalyst |

It should be noted that compounds X and Y, identical, are combined in mixture A′ (cf. (1))

3/Crosslinking in the Presence of Peroxide:

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one amphiphilic polymer, the kit comprising one or more compounds X, one or more compounds Y, and at least one peroxide, with at least one of compounds X and Y being a silicone compound and said compounds X and Y being capable of reacting together by a crosslinking reaction in the presence of a peroxide when they are brought into contact with one another, and in which the compounds X, Y and the peroxide are not present simultaneously in the same composition.

This reaction is preferably effected by heating to a temperature greater than or equal to 50° C., preferably greater than or equal to 80° C., and up to 120° C.

Compounds X and Y, which may be identical or different, have in this case at least two —CH₃ side groups and/or at least two side chains bearing a —CH₃ group.

Compounds X and Y are preferably silicone compounds and can be selected for example from the non-volatile linear polydimethylsiloxanes of high molecular weight, having a degree of polymerization above 6 and with at least two —CH₃ side groups attached to the silicon atom and/or at least two side chains bearing a —CH₃ group. We may mention for example the polymers described in the Catalogue “Reactive Silicones” of the company Gelest Inc., Edition 2004, page 6, and notably the copolymers (also called gums) of vinylmethylsiloxane-dimethylsiloxane of molecular weight in the range from 500 000 to 900 000 and notably with viscosity above 2 000 000 cSt.

As peroxides that can be used in the invention, we may mention benzoyl peroxide, 2,4-dichlorobenzoyl peroxide and mixtures thereof.

According to one embodiment, the hydrosilylation reaction in the presence of a catalyst, or the condensation reaction, or alternatively the crosslinking reaction in the presence of a peroxide, between compounds X and Y is accelerated by supply of heat, for example by raising the temperature of the system between 25° C. and 180° C.

In general, regardless of the type of reaction by which compounds X and Y react with one another, the molar percentage of X relative to the total of compounds X and Y, i.e. the ratio X/(X+Y)×100, can vary from 5 to 95%, preferably from 10 to 90%, and more preferably from 20 to 80%.

Similarly, the molar percentage of Y relative to the total of compounds X and Y, i.e. the ratio Y/(X+Y)×100, can vary from 5 to 95%, preferably from 10 to 90%, and more preferably from 20 to 80%.

Compound X can have a weight-average molecular weight (Mw) in the range from 150 to 1 000 000, preferably from 200 to 800 000, more preferably from 200 to 250 000.

Compound Y can have a weight-average molecular weight (Mw) in the range from 200 to 1 000 000, preferably from 300 to 800 000, more preferably from 500 to 250 000.

Compound X can represent from 0.1 to 95 wt. % relative to the total weight of the composition containing it, preferably from 1 to 90%, and more preferably from 5 to 80%.

Compound Y can represent from 0.1 to 95 wt. % relative to the total weight of the composition containing it, preferably from 1 to 90%, and more preferably from 5 to 80%.

The ratio of compound X to compound Y can be varied so as to adjust the reaction rate and therefore the rate of formation of the film or alternatively so as to adapt the properties of the film formed (for example its adhesive properties) according to the intended application.

In particular, compounds X and Y can be present at a molar ratio X/Y in the range from 0.05 to 20 and preferably from 0.1 to 10.

Compounds X and Y can advantageously be combined with at least one filler. Thus, the kit according to the invention can for example include, in at least one of the compositions, a filler selected from silica or surface-treated silica.

As pointed out previously, according to one embodiment of the invention, compounds X and Y can be used in the form of a single composition which then contains at least one of them or, if applicable, the catalyst or the peroxide if necessary for their interaction, in an encapsulated form.

Within the scope of the present invention, consideration is given more particularly to the encapsulated forms of the core/shell type, also called microcapsules or nanocapsules, in which the shell is of polymeric character and the core contains compound X, compound Y, one of its compounds X and Y possibly being encapsulated with the catalyst or the peroxide if necessary for the interaction of the two compounds. In the case when this catalyst is not encapsulated with one or other of the compounds X or Y, it is present in the cosmetic composition containing the encapsulated forms.

Numerous techniques are currently available for making microcapsules or nanocapsules of this type.

However, according to a preferred embodiment, the encapsulated forms considered according to the invention are nanocapsules and are obtained by a technique called solvent nanoprecipitation, notably described in documents EP 274 961 and EP 1 552 820.

More particularly, the shell of the nanocapsules of compound X or Y, employed according to the invention, is of polymeric character, not crosslinked, not water-soluble and not soluble in the capsule core.

In general, all the polymers, of natural or synthetic origin, soluble in a solvent that is not miscible with water, and notably those having a melting point below the boiling point of water at atmospheric pressure (100° C.), may be suitable.

These polymers can be biodegradable, for example polyesters, or non-biodegradable.

By way of illustration of polymers that are suitable for the invention, we may notably mention:
C₂-C₁₂ alkyl cyanoacrylate polymers
polymers formed by poly-L-lactides, poly-DL-lactides, polyglycolides and the corresponding copolymers,
polycaprolactones,
polymers of 3-hydroxybutyric acid,
copolymers of vinyl chloride and vinyl acetate,
copolymers of methacrylic acid and methacrylic ester, notably of methacrylic acid and of methacrylate,
polyvinyl acetophthalate,
cellulose acetophthalate,
polyvinylpyrrolidone-vinyl acetate copolymer,
polyethylenevinyl acetates,
polyacrylonitriles,
polyacrylamides,
polyethylene glycols,
poly-(C₁ to C₄ hydroxyalkyl methacrylate)
esters of cellulose and C₁-C₄ carboxylic acid,
polystyrene and copolymers of styrene and maleic anhydride, copolymers of styrene and acrylic acid, styrene ethylene/butylene-styrene block terpolymers, styrene-ethylene/propylene-styrene block terpolymers,
styrene alkyl-alcohol oligomers,
terpolymers of ethylene, vinyl acetate and maleic anhydride,
polyamides,
polyethylenes,
polypropylenes,
organopolysiloxanes including polydimethylsiloxanes,
poly(alkylene adipate),
polyol polyesters,
polysilsesquioxane silicone polymers,
dendritic polyesters with a hydroxyl terminal function,
polymers that are water-dispersible but are nevertheless soluble in solvents that are not miscible with water, for example: polyesters, poly(ester amides), polyurethanes and vinyl copolymers bearing carboxylic and/or sulphonic acid functions and in particular those described in document FR 2 787 729,
block copolymers insoluble in water at room temperature and solid at room temperature, having at least one block of one of the aforementioned polymers, and
mixtures thereof.

These polymers or copolymers can have a weight-average molecular weight between 1000 and 500 000 and in particular between 1500 and 100 000.

The following are quite particularly suitable for the invention: poly(alkylene adipate), organopolysiloxanes, polycaprolactones, cellulose acetophthalate, cellulose acetobutyrate, cellulose esters, polystyrene and its derivatives, and notably polycaprolactones.

Of course, a person skilled in the art is able, on the basis of his knowledge, to adjust the molecular weight of the polymer selected with respect to its concentration in the solvent so as have a mixture viscosity compatible with satisfactory emulsification.

With regard to the lipophilic core, it can contain at least one oil, in addition to compound X or compound Y. Said oil can be selected from the oils described hereunder for the oily phase. The oil is preferably a silicone oil.

According to a variant of the invention, the encapsulated forms of compound X or compound Y can be coated with a lamellar phase.

Regarding the operating procedure for production of nanocapsules suitable for the invention, a person skilled in the art can notably refer to the teaching in document EP 1 552 820 cited previously. The choice of the necessary surfactants as well as the carrying out of the method requires the knowledge of a person skilled in the art.

Physiologically Acceptable Medium

The compositions according to the invention can include a physiologically acceptable medium, i.e. a non-toxic medium that can be applied on keratinous substances of human beings and is of a pleasant appearance, odour and feel.

When the compositions according to the invention are in the form of an emulsion, the latter can be of the type of simple emulsions obtained by dispersion of a fatty phase in an aqueous phase or alternatively of the multiple emulsion type. More particularly it is a simple emulsion. These compositions are prepared according to the usual methods.

Thus, the compositions according to the invention can advantageously be in the form of an emulsion obtained by dispersion of an aqueous phase in a fatty phase (W/O) or of a fatty phase in an aqueous phase (O/W), of liquid or semi-liquid consistency of the milk type, or of soft, semi-solid or solid consistency of the cream or gel type, or alternatively a multiple emulsion (W/O/W or O/W/O). These compositions are prepared according to the usual methods.

Definition of Oily Phase:

At least one of the first and second compositions can comprise a liquid fatty phase.

The liquid fatty phase can comprise at least one oil and in particular an oil selected from hydrocarbon oils and/or silicone oils and/or fluorinated oils.

As examples of oils for use in the composition according to the invention, we may mention:
hydrocarbon oils of animal origin, such as perhydrosqualene;
hydrocarbon oils of vegetable origin, such as liquid triglycerides of fatty acids having from 4 to 10 carbon atoms such as triglycerides of heptanoic or octanoic acids or alternatively, for example sunflower oil, maize oil, soya oil, cucurbit oil, grapeseed oil, sesame oil, hazelnut oil, apricot oil, macadamia oil, arara oil, castor oil, avocado oil, triglycerides of caprylic/capric acids such as those sold by the company Stearineries Dubois or those sold under the designations Miglyol 810, 812 and 818 by the company Dynamit Nobel, jojoba oil, shea butter oil;
synthetic esters and ethers, notably of fatty acids, such as the oils of formulae R₁COOR₂ and R₁OR₂ in which R₁ represents the residue of a fatty acid having from 8 to 29 carbon atoms, and R₂ represents a linear or branched hydrocarbon chain, containing from 3 to 30 carbon atoms, for example purcelline oil, isononyl isononanoate, isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate, heptanoates, octanoates, decanoates of fatty alcohols; polyol esters, such as propylene glycol dioctanoate, neopentylglycol diheptanoate and diethylene glycol diisononanoate; and esters of pentaerythritol such as pentaerythrityl tetraisostearate;
linear or branched hydrocarbons, of mineral or synthetic origin, such as volatile or non-volatile paraffin oils, and derivatives thereof, isohexadecane, isododecane, petroleum jelly, polydecenes, hydrogenated polyisobutene such as Parleam® oil;
natural or synthetic essential oils such as, for example, eucalyptus oil, hybrid lavender oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geraniol, cade oil and bergamot oil;
fatty alcohols having from 8 to 26 carbon atoms, such as cetyl alcohol, stearyl alcohol and their mixture (cetylstearyl alcohol), octyl dodecanol, 2-butyloctanol, 2-hexyldecanol, 2-undecylpentadecanol, oleic alcohol or linoleic alcohol;
fluorinated oils, partially hydrocarbon-containing and/or silicone-containing, such as those described in document JP-A-2-295912;
silicone oils such as volatile or non-volatile polymethylsiloxanes (PDMS) with linear or cyclic silicone chain, liquid or pasty at room temperature, notably cyclopolydimethylsiloxanes (cyclomethicones) such as cyclohexasiloxane and cyclopentasiloxane; polydimethylsiloxanes bearing alkyl, alkoxy or phenyl groups, pendant or at the end of the silicone chain, groups having from 2 to 24 carbon atoms; phenylated silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
mixtures thereof.

In the list of oils given above, “hydrocarbon oil” means any oil comprising mainly carbon atoms and hydrogen atoms, and optionally ester groups, ether groups, fluorine, carboxylic acid and/or alcohol.

The other fats that can be present in the oily phase are for example fatty acids having from 8 to 30 carbon atoms, such as stearic acid, lauric acid, palmitic acid and oleic acid; waxes such as lanolin, beeswax, carnauba wax or candelilla wax, paraffin wax, lignite wax or microcrystalline waxes, ceresine or ozokerite, synthetic waxes such as polyethylene waxes, Fischer-Tropsch waxes; gums such as silicone gums (dimethiconol). These fats can be selected variously by a person skilled in the art in order to prepare compositions having desired properties, for example of consistency or texture. In particular they must be compatible with formulation of the compositions as an emulsion.

The compositions according to the invention can additionally contain a volatile oil. By “volatile oil”, we mean in the sense of the invention an oil that can evaporate in contact with keratinous substances in less than an hour, at room temperature and atmospheric pressure. The volatile oils of the invention are volatile cosmetic oils, liquid at room temperature, having a non-zero vapour pressure, at room temperature and atmospheric pressure, ranging in particular from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

As volatile oils, we may mention, among others, the cyclic or linear silicones containing from 2 to 6 silicon atoms, such as cyclohexasiloxane, dodecamethylpentasiloxane, decamethyltetrasiloxane, butyltrisiloxane and ethyltrisiloxane. It is also possible to use branched hydrocarbons, for example isododecane, as well as volatile perfluoroalkanes such as dodecafluoropentane and tetradecafluorohexane, sold under the designations “PF 5050®” and “PF 5060®” by the company 3M and the derivatives of perfluoromorpholine, such as 4-trifluoromethyl perfluoromorpholine sold under the designation “PF 5052®” by the company 3M.

The first and second compositions can be free from volatile oil.

The oil or oils can be present in the compositions according to the invention, in particular when the composition is an emulsion with aqueous continuous phase, at a content ranging from 2 to 60 wt. %, preferably from 2 to 50 wt. % relative to the total weight of the composition. As stated previously, the compositions of the emulsion type contain at least, as emulsifier, an amphiphilic polymer.

Amphiphilic Polymer

The amphiphilic polymer according to the invention can be a water-soluble or water-dispersible amphiphilic polymer or a fat-soluble or fat-dispersible amphiphilic polymer. In particular, the polymer is water-soluble or water-dispersible, notably when the composition is an emulsion with an aqueous continuous phase.

By “water-soluble or water-dispersible amphiphilic polymer”, we mean a polymer which, when added to an aqueous solution at 0.05% (by weight), can reduce the surface tension of water at 25° C. to a value below 50 mN/m, and preferably below 40 mN/m.

By “fat-soluble or fat-dispersible amphiphilic polymer”, we mean a polymer which, when added to an oil at 0.05% (by weight), can reduce its surface tension at 25° C. by at least 5 mN/m, and preferably by at least 10 mN/m.

The weight ratio of oil(s) to polymer, in particular when the composition is an emulsion with an aqueous continuous phase, is preferably between 2 and 80 or even between 5 and 50.

### 1) Water-Soluble or Water-Dispersible Amphiphilic Polymer

By “water-soluble or water-dispersible polymer”, we mean a polymer which, when added to water at a concentration of 1%, leads to a macroscopically homogeneous solution whose light transmittance, at a wavelength equal to 500 nm, through a sample with thickness of 1 cm, is at least 10%, which corresponds to an absorbance [abs=−log(transmittance)] below 1.5.

The polymers of the invention can be block polymers, or graft polymers, having on the one hand at least one water-soluble or water-dispersible polymer block and on the other hand at least one hydrophobic block.

The polymers used within the scope of the invention can therefore be block polymers, comprising for example water-soluble blocks alternating with hydrophobic blocks.

These polymers can also be in the form of graft polymers with a water-soluble or water-dispersible backbone, and bearing hydrophobic grafts. This structure can be partially crosslinked.

### Water-Soluble Polymer Blocks

By “water-soluble blocks”, we mean blocks which, when added to water at a concentration of 1%, lead to a macroscopically homogeneous solution whose light transmittance, at a wavelength equal to 500 nm, through a sample with a thickness of 1 cm, is at least 10%, which corresponds to an absorbance [abs=−log(transmittance)] below 1.5.

These water-soluble blocks can be obtained by radical polymerization of vinylic monomers, or by polycondensation, or alternatively can be constituted of existing natural polymers or modified natural polymers.

As an example, we may mention the following water-soluble monomers and their salts, which can be used to form said water-soluble or water-dispersible units, on their own or mixed:
(meth)acrylic acid,
vinylsulphonic acid and (meth)allylsulphonic acid,
vinylphosphonic acid,
methyl vinylimidazolium chloride,
(meth)acrylamide,
2-vinylpyridine and 4-vinylpyridine,
maleic acid and anhydride;
crotonic acid,
itaconic acid;
vinyl alcohol of formula CH₂═CHOH;
N-vinyllactams such as N-vinylpyrrolidone, N-vinylcaprolactam and N-butyrolactam;
water-soluble derivatives of styrene, notably styrene sulphonate;
dimethyldiallyl ammonium chloride;
N-vinylacetamide and N-methyl N-vinylacetamide,
N-vinylformamide and N-methylvinylformamide,
vinylic water-soluble monomers of the following formula (1):

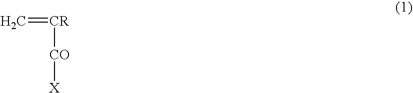

in which:
R is selected from H, —CH₃, —C₂H₅ or —C₃H₇
X is selected from:
   alkyl oxides of type —OR′ where R′ is a linear or branched, saturated or unsaturated hydrocarbon radical, having from 1 to 6 carbons, substituted with at least one halogen atom (iodine, bromine, chlorine, fluorine); a sulphonic group (—SO₃⁻), sulphate (—SO₄⁻), phosphate (—PO₄H₂⁻); hydroxy (—OH); ether (—O—); primary amine (—NH₂); secondary (—NHR₁), tertiary (—NR₁R₂) or quaternary amine (—N⁺R₁R₂R₃) with R₁, R₂ and R₃ being, independently of one another, a linear or branched, saturated or unsaturated hydrocarbon radical having 1 to 6 carbon atoms, provided that the sum of the carbon atoms of R′+R₁+R₂+R₃ does not exceed 7. We may mention quaternized dimethylaminoethyl methacrylate (MADAME), glycidyl(meth)acrylate, hydroxyethyl methacrylate, and (meth)acrylates of ethylene glycol, of diethylene glycol or of polyalkylene glycol,
   the groups —NH₂, —NHR′ and —NR′R″ in which R′ and R″ are, independently of one another, linear or branched, saturated or unsaturated hydrocarbon radicals having 1 to 6 carbon atoms, provided that the total number of carbon atoms of R′+R″ does not exceed 7, said R′ and/or R″ being substituted with a halogen atom (iodine, bromine, chlorine, fluorine); a hydroxy group (—OH); ether (—O—), sulphonic (—SO₃⁻); sulphate (—SO₄⁻); phosphate (—PO₄H₂); primary amine (—NH₂); secondary (—NHR₁), tertiary (—NR₁R₂) and/or quaternary amine (—N⁺R₁R₂R₃) with R₁, R₂ and R₃ being, independently of one another, a linear or branched, saturated or unsaturated hydrocarbon radical having 1 to 6 carbon atoms, provided that the sum of the carbon atoms of R′+R″+R₁+R₂+R₃ does not exceed 7. We may mention N,N-dimethylacrylamide, acrylamido-2-methylpropane sulphonic acid (AMPS), (meth)acrylamidopropyl trimethyl ammonium chloride (APTAC and MAPTAC).

The water-soluble blocks can also be obtained from hydrophobic monomers, said hydrophobic monomers being present in a sufficiently small amount so that these units are water-soluble. We may mention for example, as hydrophobic monomers:
styrene and its derivatives such as 4-butylstyrene, alpha-methylstyrene and vinyltoluene,
vinyl acetate of formula CH₂═CH—OCOCH₃;
vinyl ethers of formula CH₂═CHOR in which R is a linear or branched, saturated or unsaturated hydrocarbon radical, having from 1 to 6 carbons;
acrylonitrile,
caprolactone,
vinyl chloride and vinylidene chloride,
silicone-containing derivatives such as methacryloxypropyltris(trimethylsiloxy)silane and silicone-containing methacrylamides,
vinylic hydrophobic monomers of the following formula (2):

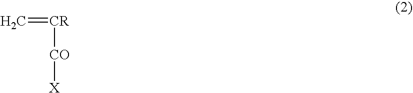

in which:
R is selected from H, —CH₃, —C₂H₅ or —C₃H₇
X is selected from:
   alkyl oxides of type —OR′ where R′ is a linear or branched, saturated or unsaturated hydrocarbon radical, having from 1 to 6 carbon atoms;
   the groups —NH₂, —NHR′ and —NR′R″ in which R′ and R″ are, independently of one another, linear or branched, saturated or unsaturated hydrocarbon radicals having 1 to 6 carbon atoms, provided that the total number of carbon atoms of R′+R″ does not exceed 6. We may mention, for example, methyl methacryiate, ethyl methacrylate, n-butyl(meth)acrylate, tert-butyl(meth)acrylate, cyclohexyl acrylate and isobornyl acrylate and ethyl 2-hexyl acrylate.

The water-soluble blocks can be non-neutralized or they can be neutralized totally or partially by an inorganic or organic base. This base can be selected, for example, from the salts of sodium, ammonium, lithium, calcium, magnesium, ammonium substituted with 1 to 4 alkyl groups bearing from 1 to 15 carbon atoms, or alternatively from mono-, di-, and triethanolamine, aminoethylpropanediol, N-methyl-glucamine, and basic amino acids, such as arginine and lysine, and mixtures thereof.

The backbone of the graft polymers can be crosslinked or non-crosslinked. It is preferably non-crosslinked.

The crosslinking agents can be selected from the compounds with olefinic polyunsaturation commonly used for the crosslinking of polymers obtained by radical polymerizations. We may mention for example divinylbenzene, diallyl ether, dipropylene glycol-diallylether, polyglycol-diallylethers, triethylene glycol-divinylether, hydroquinone-diallyl-ether, tetraallyl-oxethanoyl or other polyfunctional allyl- or vinyl ether alcohols, tetraethylene glycol diacrylate, triallylamine, trimethylolpropane-diallylether, a compound with olefinic polyunsaturation selected from divinylbenzene, tetraallyloxyethane, methylene-bis-acrylamide, allyl ethers of alcohols of the sugar series, methylene-bis-acrylamide, allyl methacrylate, trimethylol propane triacrylate (TMPTA) or mixtures thereof.

Among the polycondensates and the natural or modified natural polymers that can make up all or part of the water-soluble blocks, we may mention:
water-soluble polyurethanes,
polyethyleneimine,
polyethers such as polyoxyethylene and polyoxypropylene,
xanthan gum, notably that marketed under the designations Keltrol T® and Keltrol SF® by Kelco; or Rhodigel SM® and Rhodigel 200® from Rhodia;
alginates (Kelcosol® of Monsanto) and derivatives thereof such as alginate of propylene glycol (Kelcoloid LVF® from Kelco);
carrageenans of the iota, kappa and lambda type,
pectins of various degrees of modification (HM and LM),
cellulose derivatives and notably carboxymethylcellulose (Aquasorb A500®, Hercules), hydroxymethylcellulose, hydroxypropylcellulose, hydroxypropylmethylcellulose, hydroxyethylcellulose and quaternized hydroxyethylcellulose;
galactomannans and derivatives thereof, such as gum Konjac, gellan gum, carob gum, fennugreek gum, gum karaya, gum tragacanth, gum arabic, acacia gum, guar gum, hydroxypropylguar, hydroxypropylguar modified by sodium methylcarboxylate groups (Jaguar XC97-1®, Rhodia), guar hydroxypropyl tri-methyl ammonium chloride;
starch derivatives.

The water-soluble polymer blocks have an average molecular weight between 1000 g/mol and 10 000 000 g/mol when they constitute the water-soluble backbone of a graft polymer.

These water-soluble blocks preferably have a molecular weight between 500 g/mol and 500 000 g/mol when they constitute a block of a multiblock polymer.

Hydroyhobic Blocks

By “hydrophobic blocks”, we mean fat-soluble or fat-dispersible blocks that are alkanes, esters, ethers, triglycerides and/or silicones, fluorinated or a mixture of the oils mentioned previously.

These blocks, added to an oil or an oily mixture at a concentration of 1%, stirred at 50° C. for 48 hours, lead, after returning to room temperature, to a macroscopically homogeneous solution whose light transmittance, at a wavelength equal to 500 nm, through a sample with a thickness of 1 cm, is at least 10%, and preferably at least 20%. The oil in question can be of the alkane, ester, triglyceride, ether, silicone or fluorinated oil type or a mixture of the oils mentioned previously.

We may mention for example hydrophobic blocks comprising:
an alkyl radical having from 6 to 30 carbon atoms,
a C₆-C₃₀ fluoro or alkylfluoro radical (for example the group of formula —(CH₂)₂—(CF₂)₉—CF₃)),
a cholesteryl radical or a radical derived from cholesterol (for example cholesteryl hexanoate),
one or more cyclic aromatic group(s) such as benzene, naphthalene or pyrene,
a silicone or alkylsilicone or alternatively alkylfluorosilicone radical.

The molecular weight of these hydrophobic blocks is between 100 and 10 000 g/mol, preferably between 200 and 5000 g/mol.

The proportion by weight of the hydrophobic blocks in the final polymer is preferably between 1% and 60%, notably between 2% and 40%, and particularly between 5% and 30 wt. %, relative to the final polymer.

Synthesis

The polymers used within the scope of the invention can be prepared easily by various methods. Thus, the graft polymers can be prepared by copolymerization. This method comprises copolymerizing for example a macromonomer bearing a hydrophobic block (hydrophobic block previously described with a vinylic end group) and a water-soluble vinylic monomer such as acrylic acid or the vinylic monomers of formula (1).

The block polymers can be prepared by a coupling reaction, by living polymerization or by controlled radical polymerization. Thus, when the final polymer is in the form of a block polymer, it is possible to prepare it by a coupling reaction between water-soluble blocks and hydrophobic blocks having complementary reactive sites at each end.

It is also possible to prepare the polymers of the invention by living polymerization of the anionic or cationic type, or by controlled radical polymerization. The latter method of synthesis can be employed following various methods, for example the atom transfer route (Atom Transfer Radical Polymerization or ATRP), the radical method such as with nitroxides or alternatively the reversible chain transfer route with addition-fragmentation (Radical Addition-Fragmentation Chain Transfer) such as the MADIX method (Macromolecular Design via the Interchange of Xanthate). These methods of synthesis can be used for obtaining the water-soluble blocks and the hydrophobic blocks of the polymers of the invention; they can also be used for synthesizing just one of the two types of blocks of the polymer of the invention, the other block being introduced in the final polymer via the initiator used or alternatively by a coupling reaction between the water-soluble blocks and the hydrophobic blocks.

The water-soluble or water-dispersible amphiphilic polymer can be selected from polymers derived from acrylamido-2-methylpropane sulphonic acid (AMPS), polymers derived from acrylic acid, polyether derivatives, amphiphilic polymers obtained from natural polymers and mixtures thereof.

The polymers according to the invention, notably the polymers derived from AMPS and the polymers derived from acrylic acid, generally have a weight-average molecular weight in the range from 50 000 to 10 000 000, more preferably from 100 000 to 8 000 000 and even more preferably from 200 000 to 3 000 000.

The polymers according to the invention, notably the polymers derived from AMPS and the polymers derived from acrylic acid, are preferably neutralized partially or completely with an inorganic base (for example sodium hydroxide, potassium hydroxide, ammonia) or an organic base such as mono-, di- and tri-ethanolamine, an aminomethylpropanediol, N-methyl-glucamine, basic amino acids such as arginine and lysine, and mixtures thereof.

The amphiphilic polymers, notably polymers derived from AMPS and polymers derived from acrylic acid, according to the invention can be crosslinked or non-crosslinked; preferably, they are non-crosslinked.

a) Polymers Derived from AMPS (AcrylamidoMethylPropane Sulphonic)

The water-soluble or water-dispersible amphiphilic polymer according to the invention is notably a polymer derived from AMPS. The polymers derived from AMPS comprise:
from 80 to 99 mol. % of 2-acrylamido 2-methylpropane sulphonic (AMPS) acid unit of the following formula (3):

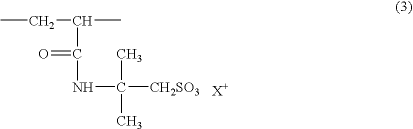

in which X⁺ is a proton, an alkali metal cation, an alkaline-earth cation or the ammonium ion; and
from 1 to 20 mol. %, and preferably from 1 to 15 mol. % of unit of the following formula (4):

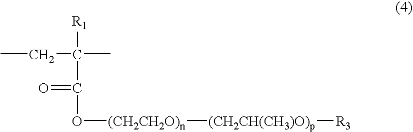

in which n and p, independently of one another, denote a number of moles and vary from 0 to 30, preferably from 1 to 20 provided that n+p is less than or equal to 30, preferably less than 25 and more preferably less than 20; R₁ denotes a hydrogen atom, a linear or branched C₁-C₆ alkyl radical (preferably methyl) and R₃ denotes a linear or branched alkyl having m carbon atoms, with m in the range from 6 to 30, preferably from 10 to 25.

The amphiphilic polymers used according to the invention can be obtained by the conventional methods of radical polymerization in the presence of one or more initiators such as for example azobisisobutyronitrile (AIBN), azobisdimethylvaleronitrile, 2,2-azobis-[2-amidinopropane]hydrochloride (ABAH), organic peroxides such as dilauryl peroxide, benzoyl peroxide, tert-butyl hydroperoxide, etc., inorganic peroxide compounds such as potassium persulphate or ammonium persulphate, or H₂O₂ optionally in the presence of reducing agents.

The polymers are notably obtained by radical polymerization in a tert-butanol medium, in which they are precipitated. By using polymerization in tert-butanol, it is possible to obtain a particle size distribution of the polymer that is particularly favourable for its uses.

The polymerization reaction can be carried out at a temperature between 0° C. and 150° C., preferably between 20° C. and 100° C., either at atmospheric pressure, or at reduced pressure. It can also be carried out under an inert atmosphere, and preferably under nitrogen.

According to this method, the polymer can be prepared starting from 2-acrylamido-2-methylpropane-sulphonic acid (AMPS) or one of its sodium or ammonium salts, with an ester of (meth)acrylic acid and:
a C₁₀-C₁₈ alcohol oxyethylenated with 8 moles of ethylene oxide (Genapol C-080® from the company Clariant),
a C₁₁ oxo alcohol oxyethylenated with 8 moles of ethylene oxide (Genapol UD-080® from the company Clariant),
a C₁₁ oxo alcohol oxyethylenated with 7 moles of ethylene oxide (Genapol UD-0700 from the company Clariant),
a C₁₂-C₁₄ alcohol oxyethylenated with 7 moles of ethylene oxide (Genapol LA-070® from the company Clariant),
a C₁₂-C₁₄ alcohol oxyethylenated with 9 moles of ethylene oxide (Genapol LA-090® from the company Clariant),
a C₁₂-C₁₄ alcohol oxyethylenated with 11 moles of ethylene oxide (Genapol LA-110® from the company Clariant),
a C₁₆-C₁₈ alcohol oxyethylenated with 8 moles of ethylene oxide (Genapol T-0800 from the company Clariant),
a C₁₆-C₁₈ alcohol oxyethylenated with 11 moles of ethylene oxide (Genapol T-110® from the company Clariant),
a C₁₆-C₁₈ alcohol oxyethylenated with 15 moles of ethylene oxide (Genapol T-150 from the company Clariant),
a C₁₆-C₁₈ alcohol oxyethylenated with 20 moles of ethylene oxide (Genapol T-200® from the company Clariant),
a C₁₆-C₁₈ alcohol oxyethylenated with 25 moles of ethylene oxide (Genapol T-250® from the company Clariant),
a C₁₈-C₂₂ alcohol oxyethylenated with 25 moles of ethylene oxide,
an iso-C₁₆-C₁₈ alcohol oxyethylenated with 25 moles of ethylene oxide.

According to a preferred embodiment, the amphiphilic polymer is a copolymer of AMPS and of C₁₆-C₁₈ alcohol methacrylate having from 6 to 25 oxyethylenated groups, obtained from methacrylic acid or from a salt of methacrylic acid and a C₁₆-C₁₈ alcohol oxyethylenated with 6 to 25 moles of ethylene oxide.

The amphiphilic polymer can also be a copolymer of AMPS and of C₁₂-C₁₄ alcohol methacrylate having from 6 to 25 oxyethylenated groups, obtained from methacrylic acid or from a salt of methacrylic acid and a C₁₂-C₁₄ alcohol oxyethylenated with 6 to 25 moles of ethylene oxide.

As amphiphilic polymers that are preferred according to the present invention, we may mention:
the non-crosslinked copolymer obtained from 92.65 mol. % of AMPS and of 7.35 mol. % of C₁₆-C₁₈ alcohol methacrylate bearing 8 oxyethylenated groups (Genapol T-080®),
the non-crosslinked copolymer obtained from 91.5 mol. % of AMPS and 8.5 mol. % of C₁₂-C₁₄ alcohol methacrylate bearing 7 oxyethylenated groups (Genapol LA-070®),
the crosslinked copolymer obtained from 96.45 mol. % of AMPS and 3.55 mol. % of C₁₆-C₁₈ alcohol methacrylate bearing 25 oxyethylenated groups (Genapol T-250®), the crosslinking agent being trimethylol propane triacrylate,
the crosslinked copolymer obtained from AMPS and C₂₋₂ alcohol methacrylate bearing 25 oxyethylenated groups; this polymer is sold under the designation Aristoflex HMB® by the company Clariant.

These copolymers are suitable for providing stable emulsions and can have very varied textures, ranging from sprayable fluid to cream with very good cosmetic characteristics.

Even more preferably, the amphiphilic polymers of the invention are selected from:
the non-crosslinked copolymer obtained from 92.65 mol. % of AMPS and 7.35 mol. % of C₁₆-C₁₈ alcohol methacrylate bearing 8 oxyethylenated groups (Genapol T-080®),
the non-crosslinked copolymer obtained from 91.5 mol. % of AMPS and 8.5 mol. % of C₁₂-C₁₄ alcohol methacrylate bearing 7 oxyethylenated groups (Genapol LA-070®),
and mixtures thereof.

b) Polymers Derived from Acrylic Acid

The amphiphilic polymer according to the invention can be selected from the derivatives of acrylic acid. These polymers comprise:
from 80 to 99 mol. % of acrylic acid (AA) acid unit of the following formula (5):

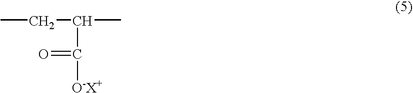

in which X⁺ is a proton, an alkali metal cation, an alkaline-earth cation or the ammonium ion; and
from 1 to 20 mol. %, and preferably from 1 to 15 mol. % of unit of the following formula (6):

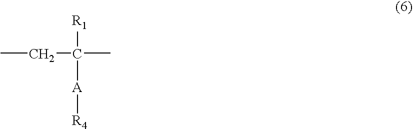

in which R₁ denotes a hydrogen atom, a linear or branched C₁-C₆ alkyl radical (preferably methyl), A denotes an ester or amide group or an oxygen atom and R4 denotes a linear or branched alkyl having m carbon atoms with m in the range from 6 to 30, preferably from 10 to 25.

As amphiphilic polymers derived from acrylic acid that are preferred according to the present invention, we may mention:
the non-crosslinked copolymer obtained from (meth)acrylic acid and steareth-20 methacrylate, sold under the designation Aculyn 22® by the company Rohm and Haas,
the non-crosslinked copolymer obtained from (meth)acrylic acid and laureth-25 methacrylate, sold under the designation Aculyn 25® by the company Rohm and Haas,
the non-crosslinked copolymer obtained from (meth)acrylic acid and beheneth-25 methacrylate, sold under the designation Aculyn 28® by the company Rohm and Haas,
the crosslinked copolymer obtained from (meth)acrylic acid and vinyl neodecanoate, sold under the designation Aculyn 38® by the company Rohm and Haas,
the crosslinked copolymer obtained from (meth)acrylic acid and steareth-20 methacrylate, sold under the designation Aculyn 880 by the company Rohm and Haas,
the crosslinked copolymers of alkyl-C₁₀-C₃₀ acrylate and (meth)acrylic acid, such as Pemulen TR10 and TR2® sold by the company Noveon,
the crosslinked copolymer of acrylic acid and vinyl iso-decanoate, sold under the designation Stabylen 30® by the company 3V,
the crosslinked copolymers obtained from (meth)acrylic acid and C₁₀-C₃₀ alkyl acrylate, sold under the designation Carbopol ETD 2020® and Carbopol 1382® by the company Noveon,
the non-crosslinked copolymer obtained from (meth)acrylic acid and steareth-20 itaconate, sold under the designation Structure 2001® by the company National Starch,
the non-crosslinked copolymer obtained from (meth)acrylic acid and ceteth-20 itaconate, sold under the designation Structure 3001® by the company National Starch,
the non-crosslinked copolymer obtained from (meth)acrylic acid, aminoacrylate and C₁₀-C₃₀ alkyl PEG 20 itaconate, sold under the designation Structure Plus® by the company National Starch,
the non-crosslinked copolymer obtained from (meth)acrylic acid, methylacrylate and dimethyl meta-isopropenyl benzyl isocyanate of ethoxylated alcohol, sold under the designation Viscophobe DB 1000® by the company Amerchol.

The polymer derived from acrylic acid is in particular a non-crosslinked copolymer obtained from (meth)acrylic acid, methylacrylate and dimethyl meta-isopropenyl benzyl isocyanate of ethoxylated alcohol.

c) Polyether Derivatives

The amphiphilic polymers derived from polyether, preferred in the invention, are water-soluble polyurethanes and notably polyethylene glycol compounds (for example having from 45 to 160 ethylene oxide units) bearing at the ends a C₈-C₂₀ alkyl chain via a urethane linkage.

Among the latter, we may mention:
the PEG-150 copolymer bearing stearyl end groups via urethane linkages, sold under the designation Aculyn 46® by the company Rohm and Haas,
the PEG-150 copolymer bearing decyl end groups via urethane linkages, sold under the designation Aculyn 44® by the company Rohm and Haas,
the PEG-136 copolymer bearing stearyl end groups via urethane linkages, sold under the designation Nuvis FX 1100® by the company Elementis,
the PEG-50 copolymer bearing stearyl end groups via urethane linkages, sold under the designation Borchigel LW 44® by the company Borchers France.

The polymers derived from polyether can also bear fatty chains without urethane linkages, for example the product Pure Thix HH® sold by the company Sud Chemie.

The polymers derived from polyether can also be selected from the block copolymers of ethylene oxide and propylene oxide, with the following formula:

HO(C₂H₄O)ₓ(C₃H₆O)_{y}(C₂H₄O)_{z}H

in which x, y and z are integers such that x+z is in the range from 2 to 100 and

y is in the range from 14 to 60, and more particularly from the block copolymers of the above formula having an HLB in the range from 7 to 16.

These block copolymers can notably be selected from the polyethylene glycol/polypropylene glycol/polyethylene glycol triblock polycondensates and notably from those sold under the designations:
Pluronic L42® of the above formula with x=z=3 and y=18 (HLB=8)
Pluronic P84® (INCI name: POLOXAMER 184) of the above formula with x=z=12 and y=27 (HLB=14)
Pluronic P103® (INCI name: POLOXAMER 333) of the above formula withx=z=12 andy=40(HLB=9)
Pluronic P123® (INCI name: POLOXAMER 184) of the above formula with x=z 14 and y=48 (HLB=8)
Pluronic L44® (INCI name: POLOXAMER 124) of the above formula with x=z=5 and y=21 (HLB=16).

d) Amphiphilic Polymers Obtained from Natural Polymers

The water-soluble or water-dispersible amphiphilic polymers of natural origin preferred in the invention are for example selected from cellulose derivatives, guar derivatives, starch derivatives and derivatives of acacia gum modified with fatty chains having from 6 to 30 carbon atoms.

Among the cellulose derivatives modified with fatty chains having from 6 to 30 carbon atoms we may mention:
cetylhydroxyethylcelluloses such as Natrosol CS Plus 330′, 430′ and Polysurf 67 CS® sold by the company Hercules,

hydroxypropylmethylcelluloses modified with stearyloxyhydroxypropyl chains with a molar proportion between 0.3 and 0.6%, sold under the designations Sangelose 60L® (molecular weight of the order of 500 000 g/mol) and Sangelose 90L® (molecular weight of the order of 900 000 g/mol) by the company Daido,
hydroxyethylcellulose quaternized with substituted lauryl di-methyl ammonium epoxide, sold under the designation Quadrisoft LM 2000 by the company Amerchol,
hydroxyethylcellulose quaternized and modified with lauryl or stearyl chains, sold under the designation Crodacel QM® (C12), QL® (C12) and QSO(C18) by the company Croda.

Among the guar derivatives modified with fatty chains having from 6 to 30 carbon atoms, we may mention hydroxypropylguar modified with behenic chains, sold under the designation Esaflor HM 22® by the company Lamberti.

Among the starch derivatives modified with fatty chains having from 6 to 30 carbon atoms, we may mention maize starch esterified by octenylsuccinic anhydride in the form of sodium salt, sold under the designation N-creamer 460 by the company National Starch.

Among the derivatives of acacia gum modified with fatty chains having from 6 to 30 carbon atoms, we may mention acacia gum modified by controlled esterification, sold under the designation Ticamulsion A-2010® by the company Tic Gums.

The amphiphilic polymer obtained from natural polymer is in particular a cetylhydroxyethylcellulose.

Among the water-soluble or water-dispersible amphiphilic polymers, preferably the following are used:
the non-crosslinked copolymer obtained from 92.65 mol. % of AMPS and 7.35 mol. % of C₁₆-C₁₈ alcohol methacrylate bearing 8 oxyethylenated groups (Genapol T-080® ),
the non-crosslinked copolymer obtained from 91.5 mol. % of AMPS and 8.5 mol. % of C₁₂-C₁₄ alcohol methacrylate bearing 7 oxyethylenated groups (Genapol LA-070®),
the crosslinked copolymer obtained from (meth)acrylic acid and steareth-20 methacrylate, sold under the designation Aculyn 88® by the company Rohm and Haas,
the crosslinked copolymers of alkyl-C₁₀-C₃₀ acrylate and (meth)acrylic acid, such as Pemulen TR1® and TR2® sold by the company Noveon,
the non-crosslinked copolymer obtained from (meth)acrylic acid, methylacrylate and dimethyl meta-isopropenyl benzyl isocyanate of ethoxylated alcohol, sold under the designation Viscophobe DB 1000® by the company Amerchol,
the PEG-136 copolymer bearing stearyl end groups via urethane linkages, sold under the designation Nuvis FX 1100® by the company Elementis,
the cetylhydroxyethylcellulose polymer,
and mixtures thereof.

The amphiphilic polymers more especially preferred are
the non-crosslinked copolymer obtained from 92.65 mol. % of AMPS and 7.35 mol. % of C₁₆-C₁₈ alcohol methacrylate bearing 8 oxyethylenated groups (Genapol T-080®),
the non-crosslinked copolymer obtained from (meth)acrylic acid, methylacrylate and dimethyl meta-isopropenyl benzyl isocyanate of ethoxylated alcohol, sold under the designation Viscophobe DB 1000® by the company Amerchol
the cetylhydroxyethylcellulose polymer obtained from natural polymer
and mixtures thereof.

### 2) Fat-Soluble or Fat-Dispersible Amphiphilic Polymer

By “fat-soluble or fat-dispersible polymers”, we mean polymers which, when added to an oil or an oily mixture, at a concentration of 1%, stirring at 50° C. for 48 hours, lead after returning to room temperature to a macroscopically homogeneous solution whose light transmittance, at a wavelength equal to 500 nm, through a sample with a thickness of 1 cm, is at least 10%, preferably at least 20%.

The fat-soluble or fat-dispersible amphiphilic polymers of the invention can make it possible to stabilize emulsions with an oily continuous phase.

They can be constituted of at least one polar moiety and at least one apolar moiety. They can have a structure of the block or comb type.

The fat-soluble or fat-dispersible amphiphilic polymers can be selected from derivatives of polyolefins, dimethicone copolyols, crosslinked elastomers of emulsifying organopolysiloxanes, derivatives of polyethers, derivatives of polyhydroxystearate and mixtures thereof.

a. Derivatives of Polyolefins

The amphiphilic polymers derived from polyolefins can be used for making water-in-oil emulsions that are stable in storage and suitable for cosmetic, dermatologic and/or pharmaceutical applications. In particular, they can be used for preparing water-in-oil emulsions with a very high proportion of aqueous phase (more than 80 wt. %) and stable, or alternatively water-in-oil emulsions of low viscosity and stable. These surfactants thus offer advantages relative to the emulsifiers commonly used for stabilizing water-in-oil emulsions, in particular alkyl polyglycerols, alkyl POEs, alkyl sorbitans, metal salts of fatty acids and silicone-containing surfactants. In fact, for these emulsifiers, the content of aqueous phase is generally below 80 wt. %, the concentrations of surfactants are high to ensure good stability of the emulsions and the fatty phase comprises mainly silicone oils when silicone-containing surfactants are used. Conversely, the water-in-oil emulsions stabilized with a surfactant derived from polyolefins possess particularly good cosmetic properties, with a light and fresh feel, without being siliconized.

The apolar moiety can be selected from the polyolefins such as polymers and/or copolymers of ethylene, of propylene, of 1-butene, of isobutene, of 1-pentene, of 2-methyl-1-butene, of 3-methyl-1-butene, of 1-hexene, of 1-heptene, of 1-octene, of 1-decene, of 1-undecene, of 1-dodecene, of 1-tridecene, of 1-tetradecene, of 1-pentadecene, of 1-hexadecene, of 1-heptadecene and of 1-octadecene. The polymer chains are hydrogenated or not. They are constituted of at least 40 carbons, and preferably of 60 to 700 carbons.

The polar moiety of the amphiphilic polymers of the invention can be anionic, cationic, nonionic, zwitterionic or amphoteric. It is for example constituted of acrylic derivatives, of polyalkylene glycols or of polyalkylene imine. The amphiphilic polymers with carboxylic acid polar moiety result for example from reaction of a polyolefin with carboxylic acids such as maleic acid, maleic anhydride, fumaric acid, itaconic acid, citraconic acid, mesaconic acid, aconitic acid. Preferably, their polar moiety is constituted of succinic acid or anhydride, their ester or amide derivatives, salts of alkaline ions, alkaline-earth ions, or corresponding organic derivatives, or polyoxyethylene.

The fat-soluble or fat-dispersible amphiphilic polymers of the invention are for example polyisoprene-polyoxyethylene or poly(ethylene-co-propylene)-polyoxyethylene diblock polymers described in the work of Allgaier, Poppe, Willner, Richter (Macromolecules, 1997, 30, p. 1582-1586).

The amphiphilic polymers of the invention can also be selected from the polyolefin derivatives of succinic acid described in patents U.S. Pat. No. 4,234,435, U.S. Pat. No. 4,708,753, U.S. Pat. No. 5,129,972, U.S. Pat. No. 4,931,110, GB 2 156 799 and U.S. Pat. No. 4,919,179. The polyolefin moiety can be constituted of polyisobutylene, hydrogenated or not. The succinic anhydride or acid can be modified with alcohols, amines, alkanolamines or polyols, or alternatively can be in the form of salts of alkaline or alkaline-earth ions, or alternatively of organic ions such as the diethanolammonium or triethanolammonium ions. We may notably mention polyisobutylene with modified succinic termination, such as the products marketed under the designations L2724®, L2721®, L2722®, OS156565® and Lubrizol 5603® by the company Lubrizol.

Another example of amphiphilic polymer for use in the invention is the product of the reaction of maleic anhydride with polyisobutylene, such as Glissopal ITS® marketed by BASF.

b. Dimethicone Copolyols

Among the amphiphilic polymers suitable for stabilizing emulsions with an oily continuous phase, we may mention the dimethicone copolyols such as the mixture of cyclomethicone and dimethicone copolyol, sold under the designation DC 5225 C® by the company Dow Corning, and the alkyl-dimethicone copolyols such as the laurylmethicone copolyol sold under the designation Dow Corning 5200 Formulation Aid® by the company Dow Corning and the cetyl dimethicone copolyol sold under the designation ABIL EM 90® by the company Goldschmidt, or the polyglyceryl-4 isostearate/cetyl dimethicone copolyol/hexyllaurate mixture sold under the designation ABIL WE 09® by the company Goldschmidt, or the oxyethylenated polydimethylmethylsiloxane sold under the designation DC SH 3773 M® by the company Dow Corning.

c. Crosslinked Elastomers of Emulsifying Organopolysiloxanes

It is also possible to use a crosslinked solid elastomeric organopolysiloxane having at least one oxyalkylenated group, such as those obtained according to the procedure in examples 3, 4 and 8 in document U.S. Pat. No. 5,412,004 and in the examples in document U.S. Pat. No. 5,811,487, notably the product of example 3 (example of synthesis) of patent U.S. Pat. No. 5,412,004 and such as that marketed under the reference KSG 21® by the company Shin Etsu.

It is also possible to use elastomers of emulsifying silicones such as those sold under the designations KSG-210, KSG-310, KSG-320, KSG-330, KSG-440, KSG-710, KSG-830, KSG-840 by the company SHIN-ETSU.

d. Polyether Derivatives

The polymer derived from polyether can also be selected from the block copolymers of ethylene oxide and propylene oxide, of the following formula:

HO(C₂H₄O),(C₃H₆O)_{y}(C₂H₄O)_{z}H

in which x, y and z are integers such that x+z is in the range from 2 to 100 and y is in the range from 14 to 60, and more particularly from the block copolymers of the above formula having an HLB in the range from 2 to 6.

These block copolymers can notably be selected from the polyethylene glycol/polypropylene glycol/polyethylene glycol triblock polycondensates sold under the designations “SYNPERONIC” such as “SYNPERONIC®PE/L81” (INCI name: POLOXAMER 231); “SYNPERONIC® PE/L92” (INCI name: POLOXAMER 282) by the company UNIQEMA.

e. Polyhydroxystearate Derivatives

The amphiphilic polymer according to the invention can be selected from the polymers derived from polyhydroxystearate possessing a structure of the multiblock type. Preferably, these polymers are made up of three blocks B-A-B where B is a polyhydroxystearate and A is a polyether. We may mention the copolymer of polyethylene glycol (30 OE) and of 12-hydroxy stearic acid (INCI name: PEG-30 dipolyhydroxystearate) sold under the designation Arlacel P135® by the company Uniqema.

The amphiphilic polymer contained in the kit or compositions according to the invention is preferably selected from the water-soluble or water-dispersible amphiphilic polymers, notably from those described previously.

The amphiphilic polymer can be present in at least one of the first and second compositions according to the invention, in particular the emulsions with aqueous continuous phase, at a content between 0.01 and 10%, preferably between 0.05 and 5%, more preferably between 0.25 and 3%, by weight relative to the total weight of the composition.

The amphiphilic polymer can be present in the compositions according to the invention, in particular the emulsions with oily continuous phase, at a content between 0.1 and 10%, preferably between 1 and 5%, by weight relative to the total weight of the composition.

The concentration of amphiphilic polymer and of compound X and/or of compound Y in the compositions according to the invention can be such that the weight ratio of the amphiphilic polymer to compound X and/or compound Y is between 0.01 and 1, in particular between 0.05 and 0.2.

Optionally, without it being obligatory, it is possible to add, in order to facilitate the emulsification of the oily phase, at least one co-surfactant different from the amphiphilic polymer described previously.

Surfactants

The co-surfactant(s) is/are used for example in a proportion of less than 50% relative to the weight of the amphiphilic polymer according to the invention.

This surfactant can be nonionic, amphoteric or cationic and is preferably anionic.

We may mention for example as nonionic surfactants, notably the esters of polyols and of fatty acid with a saturated or unsaturated chain having for example from 8 to 24 carbon atoms and preferably from 12 to 22 carbon atoms, and oxyalkylenated derivatives thereof, i.e. containing oxyethylenated and/or oxypropylenated units, such as the esters of glyceryl and of C₈-C₂₄ fatty acid, and oxyalkylenated derivatives thereof; the esters of polyethylene glycol and of C₈-C₂₄ fatty acid, and oxyalkylenated derivatives thereof; the esters of sorbitol and of C₈-C₂₄ fatty acid, and oxyalkylenated derivatives thereof; the esters of sugar (sucrose, glucose, alkylglucose) and of C₈-C₂₄ fatty acid, and oxyalkylenated derivatives thereof; the ethers of fatty alcohols; the ethers of sugar and of C₈-C₂₄ fatty alcohols, and mixtures thereof.

As ester of glyceryl and of fatty acid, we may notably mention glyceryl stearate (mono-, di- and/or tri-glyceryl stearate) (CTFA name: glyceryl stearate) or glyceryl ricinoleate, and mixtures thereof.

As ester of polyethylene glycol and of fatty acid, we may notably mention polyethylene glycol stearate (mono-, di- and/or tri-stearate of polyethylene glycol), and more especially polyethylene glycol monostearate 50 OE (CTFA name: PEG-50 stearate), polyethylene glycol monostearate 100 OE (CTFA name: PEG-100 stearate) and mixtures thereof.

It is also possible to use mixtures of these surfactants, for example the product containing glyceryl stearate and PEG-100 stearate, marketed under the designation ARLACEL 165 by the company Uniqema, and the product containing glyceryl stearate (mono-diglyceryl stearate) and potassium stearate, marketed under the designation TEGIN by the company Goldschmidt (CTFA name: glyceryl stearate SE).

As ester of fatty acid and of glucose or of alkylglucose, we may mention in particular glucose palmitate, alkylglucose sesquistearates such as methylglucose sesquistearate, alkylglucose palmitates such as methylglucose palmitate or ethylglucose palmitate, aliphatic esters of methylglucoside and more especially the diester of methylglucoside and oleic acid (CTFA name: methyl glucose dioleate); mixed ester of methylglucoside and of oleic acid/hydroxystearic acid mixture (CTFA name: methyl glucose dioleate/hydroxystearate); ester of methylglucoside and isostearic acid (CTFA name: methyl glucose isostearate); ester of methylglucoside and lauric acid (CTFA name: methyl glucose laurate); mixture of monoester and diester of methylglucoside and isostearic acid (CTFA name: methyl glucose sesqui-isostearate); mixture of monoester and diester of methylglucoside and stearic acid (CTFA name: methyl glucose sesquistearate) and in particular the product marketed under the designation Glucate SS by the company AMERCHOL, and mixtures thereof.

As oxyethylenated ethers of fatty acid and of glucose or of alkylglucose, we may mention for example the oxyethylenated ethers of fatty acid and of methylglucose, and in particular the polyethylene glycol ether of diester of methyl glucose and stearic acid with about 20 moles of ethylene oxide (CTFA name: PEG-20 methyl glucose distearate) such as the product marketed under the designation Glucam E-20 distearate by the company AMERCHOL; the polyethylene glycol ether of the mixture of monoester and diester of methyl glucose and stearic acid with about 20 moles of ethylene oxide (CTFA name: PEG-20 methyl glucose sesquistearate) and in particular the product marketed under the designation Glucamate SSE-20 by the company AMERCHOL and that marketed under the designation Grillocose PSE-20 by the company GOLDSCHMIDT, and mixtures thereof.

As esters of sucrose, we may mention for example sucrose palmito-stearate, sucrose stearate and sucrose monolaurate.

As ethers of fatty alcohols, we may mention for example the ethers of polyethylene glycol and of fatty alcohol having from 8 to 30 carbon atoms, and notably from 10 to 22 carbon atoms, such as the ethers of polyethylene glycol and cetyl alcohol, stearyl alcohol, cetearyl alcohol (mixture of cetyl and stearyl alcohols). We may mention for example the ethers having from 1 to 200 and preferably from 2 to 100 oxyethylenated groups, such as those of CTFA name Ceteareth-20, Ceteareth-30, and mixtures thereof.

As ethers of sugar, we may notably mention the alkylpolyglucosides, and for example decylglucoside such as the product marketed under the designation MYDOL 10 by the company Kao Chemicals, the product marketed under the designation PLANTAREN 2000 by the company Henkel, and the product marketed under the designation ORAMIX NS 10 by the company Seppic; caprylyl/capryl glucoside such as the product marketed under the designation ORAMIX CG 110 by the Company Seppic or under the designation LUTENSOL GD 70 by the Company BASF; laurylglucoside such as the products marketed under the designations PLANTAREN 1200 N and PLANTACARE 1200 by the company Henkel; coco-glucoside such as the product marketed under the designation PLANTACARE 818/UP by the company Henkel; cetostearyl glucoside optionally mixed with cetostearyl alcohol, marketed for example under the designation MONTANOV 68 by the company Seppic, under the designation TEGO-CARE CG90 by the company Goldschmidt and under the designation EMULGADE KE3302 by the company Henkel; arachidyl glucoside, for example in the form of the mixture of arachidic and behenic alcohols and of arachidyl glucoside marketed under the designation MONTANOV 202 by the company Seppic; cocoylethylglucoside, for example in the form of mixture (35/65) with cetyl and stearyl alcohols, marketed under the designation MONTANOV 82 by the company Seppic, and mixtures thereof.

Among the esters or ethers of polyols, we may mention more particularly glycerol esters, polyethylene glycol esters, sorbitan esters, and mixtures thereof.

Other suitable products for the invention include mono-isoglyceryl stearate, such as the product marketed under the designation Peceol Isostariquee by the company Gattefosse, PEG-8 isostearate such as the product marketed under the designation Prisorine 36440 by the company Uniquema, isostearate polyglycerolated (4 moles) sold under the designation Isolan G1340 by the company Goldschmidt, diisostearate polyglycerolated (3 moles) sold under the designation Lameform TGI® by the company Cognis and distearate polyglycerolated (2 moles) sold under the designation Emalex PGSA® by the company Nihon emulsion.

Sorbitan isostearate such as the product marketed under the designation Arlacel 987® by the company Uniqema, isosterate of sorbitan and of glycerol, such as the product marketed under the designation Arlacel 986® by the company Uniqema, sorbitan sesquioleate such as the product sold under the designation Arlacel 83V® by the company Uniqema, sorbitan laurate, sorbitan oleate and sorbitan trioleate such as the products marketed under the designation Span 20®, Span 80V® and Span 85V® by the company Uniqema can also be used.

As sugar esters, we may mention for example methylglucose isostearate.

According to a preferred embodiment of the invention, the coemulsifier is selected from the glyceryl esters.

The compositions of the invention can contain all of the additives usually employed in cosmetics and will find applications in the area of care, make-up, sun-tan products and body hygiene.

Thus, the aqueous phase of the compositions can contain glycols such as dipropylene glycol, glycerol and butylene glycol, water-soluble or water-dispersible organic and mineral UV filters, actives, salts, fillers. As actives, we may notably use vitamins (A, C, E, K, PP etc.) alone or mixed, as well as their derivatives, keratolytic and/or desquamating agents (salicylic acid and its derivatives, alpha-hydroxyacids, ascorbic acid and its derivatives), anti-inflammatory agents, thickeners, depigmenting agents, lifting agents such as synthetic polymers, vegetable proteins, polysaccharides of vegetable origin whether or not in the form of microgels, starches, dispersions of waxes, mixed silicates and colloidal particles of inorganic fillers; dulling agents, or alternatively anti-wrinkle agents and mixtures thereof. The oily phase can contain lipophilic gelling agents, waxes, organic or mineral particles or alternatively lipophilic or fat-dispersible organic and mineral sun filters.

Depending on the fluidity of the composition that is to be produced, one or more gelling agents, notably hydrophilic, i.e. soluble or dispersible in water, can be incorporated in the composition.

As hydrophilic gelling agents, we may mention in particular water-soluble or water-dispersible thickening polymers. The latter can notably be selected from: modified or unmodified carboxyvinyl polymers, such as the products marketed under the designations Carbopol® (CTFA name: carbomer) by the company Noveon; polyacrylates and polymethacrylates such as the products sold under the designations Lubrajel® and Norgel® by the company GUARDIAN or under the designation Hispagel® by the company HISPANO CHIMICA; polyacrylamides; polymers and copolymers of 2-acrylamido 2-methylpropane sulphonic acid, optionally crosslinked and/or neutralized, such as poly(2-acrylamido 2-methylpropane sulphonic acid) marketed by the company CLARIANT under the designation Hostacerin AMPS® (CTFA name: ammonium polyacryldimethyltauramide); crosslinked anionic copolymers of acrylamide and of AMPS, in the form of a W/O emulsion, such as those marketed under the name of SEPIGEL 305® (CTFA name: Polyacrylamide/CI₃₋₁₄ Isoparaffin/Laureth-7) and under the name of SIMULGEL 600® (CTFA name: Acrylamide/Sodium acryloyldimethyltaurate copolymer/Isohexadecane/Polysorbate 80) by the company SEPPIC; polysaccharide biopolymers such as xanthan gum, guar gum, carob gum, acacia gum, scleroglucans, derivatives of chitin and of chitosan, carrageenans, gellans, alginates, celluloses such as microcrystalline cellulose, carboxymethylcellulose, hydroxymethylcellullose and hydroxypropylcellulose; and mixtures thereof.

As lipophilic gelling agents, we may mention for example modified clays such as modified magnesium silicate (bentone gel VS38® from RHEOX), hectorite modified with distearyl dimethyl ammonium chloride (CTFA name: disteardimonium hectorite) marketed under the designation bentone 38 CE® by the company RHEOX.

The compositions according to the invention can in addition contain fillers.

As fillers, we may mention for example particles of polyamide (Nylon®) and notably those sold under the designations ORGASOL® by the company Arkema; polyethylene powders; microspheres based on acrylic copolymers, such as those made of ethylene glycol dimethacrylate/lauryl methacrylate copolymer sold by the company Dow Corning under the designation POLYTRAP®; microspheres of polymethyl methacrylate, marketed under the designation MICROSPHERE M-100® by the company Matsumoto or under the designation COVABEAD LH85® by the company Wackherr; powders of ethylene-acrylate copolymer, such as those marketed under the designation FLOBEADS® by the company Sumitomo Seika Chemicals; the expanded powders such as hollow microspheres and notably microspheres formed from a vinylidene chloride, acrylonitrile and methacrylate terpolymer, marketed under the designation EXPANCEL® by the company Kemanord Plast under the references 551 DE 12®& (grain size of about 12 μm and density 40 kg/m³), 551 DE 20® (grain size of about 30 μm and density 65 kg/m³), 551 DE 50 (grain size of about 40 μm), or the microspheres marketed under the designation MICROPEARL F 80 ED® by the company Matsumoto; powders of natural organic material such as starch powders, notably of maize starch, wheat starch or rice starch, crosslinked or not, such as powders of starch crosslinked with octenylsuccinate anhydride, marketed under the designation DRY-FLO® by the company National Starch; silicone resin microbeads such as those marketed under the designation TOSPEARL® by the company Toshiba Silicone, notably TOSPEARL 240®; and mixtures thereof.

At least one of the compositions in a kit according to the invention can also include at least one colorant.

For application in particular for the care or for make-up of greasy skin, the compositions according to the invention can include at least one active selected from: desquamating agents, anti-seborrhoeic agents, antimicrobial agents, and soothing agents.

For application in particular for the care or make-up of aged skin, the compositions according to the invention can include at least one active selected from: desquamating or hydrating agents; depigmenting or anti-pigmenting agents; antiglycating agents; anti-NO agents; agents stimulating the synthesis of dermal or epidermal macromolecules and/or preventing their degradation; agents stimulating the proliferation of fibroblasts or of keratinocytes and/or differentiation of keratinocytes; muscle relaxants or dermo-decontracting agents; anti-radical or anti-pollution agents; lifting agents; and agents acting on the microcirculation.

The compositions of this type can be in the form of a care or make-up product, a hair-care product, a sun-tan product or a body-hygiene product and can be packaged for example in the form of cream in a pot or of fluid in a tube or in a pump bottle.

The compositions according to the invention or used for the method according to the invention can be in the form of a composition for protection, treatment or care for the face, the hands, the feet, for the principal anatomic folds or for the body (for example day cream, night cream, make-up-remover cream, sunscreen composition, body milk for protection or care, after-sun milks, skin-care lotion, gel or mousse, artificial tanning composition); an after-shave composition.

The kit according to the invention can notably be used as a composition for coating the skin of the body or of the face, and more particularly a composition for make-up and/or care of the skin of the body or of the face.

The compositions according to the invention can be used for make-up of the skin, of the body and of the face, of the lips, of the eyelashes and/or of the nails, depending on the nature of the ingredients used.

In particular, the composition or compositions according to the invention can be in the form of a foundation, a lipstick or lip rouge paste, anti-eye-circle or contour product, eye-liner, eye shadow, product for make-up of the body or alternatively a skin colouring product.

A person skilled in the art will be able to select the appropriate galenical form, as well as its method of preparation, on the basis of his general knowledge, taking into account, on the one hand, the nature of the constituents used, notably their solubility in the carrier, and, on the other hand, the application envisaged for each composition.

The invention is illustrated in more detail, non-limitatively, by the examples described below. Unless stated otherwise, the quantities shown are expressed as percentage by weight.

### EXAMPLES

In the examples of compositions described hereunder, the combination of the following mixtures A and B produced by the company Dow Corning is used as compounds X and Y:

MIXTURE A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

The amphiphilic copolymers in the following examples are supplied either in the form of powder or as aqueous solution.

When they are in the form of powder, they are dissolved in water for 30 minutes with stirring at 25° C.; the solution obtained is macroscopically homogeneous.

When they are in aqueous solution, they are diluted with water for 30 minutes with stirring at 25° C.; the solution obtained is macroscopically homogeneous.
The emulsions in the following examples are prepared by slowly adding the oily phase to the aqueous phase while stirring by means of a homogenizer of the Moritz type at a stirring speed of 4000 rpm for 15 minutes.
The gloss of the emulsions after drying in the form of film is measured according to the following in vitro protocol.

Apply a film of the test composition on a contrast card (Prüfkarte type 24/5-250 cm² marketed by the company Erichsen) using a film-puller (wet thickness 50 microns). In the case of a product according to the invention, mix, at the time of use, the first and second compositions containing respectively compounds X and Y in a 50/50 weight ratio, then apply this mixture on the card. Then dry the composition for 24 hours at a temperature of 37° C.

The gloss is measured using the micro TRI gloss BYK Gardner gloss-meter. The measurement of gloss is performed by measuring the light reflected at an angle of 20° to the normal to the plane of the sample.

### Example 1

### Oil-in-Water Emulsions Containing an Amphiphilic Polymer Based on AMPS

The compositions of the emulsions are as follows:

The amphiphilic polymer used is water-soluble and has a comb structure. It is obtained from 92.65 mol. % of AMPS and 7.35 mol. % of C₁₆-C₁₈ alcohol methacrylate containing 8 oxyethylenated groups (Genapol T080) and is supplied by the company Clariant.

| | | Concentration (wt. %) | | | |
|---|---|---|---|---|---|
| | | Emulsion | Emulsion | Emulsion | Mixture |
| Phase | Name | 1a | 1b | 1c | 1a + 1b |
| A | Distilled water | 78.296 | 78.296 | 88.296 | 78.296 |
| | Preservative | 1 | 1 | 1 | 1 |
| | Triethanolamine | 0.004 | 0.004 | 0.004 | 0.004 |
| | Copolymer of AMPS and | 0.5 | 0.5 | 0.5 | 0.5 |
| | of C₁₆-C₁₈ alcohol | | | | |
| | methacrylate containing 8 | | | | |
| | oxyethylenated groups¹ | | | | |
| | Polyacrylamidomethyl | 0.2 | 0.2 | 0.2 | 0.2 |
| | propane sulphonic acid | | | | |
| | partially neutralized with | | | | |
| | ammonia and highly crosslinked² | | | | |
| B | Parleam oil | 5 | 5 | 5 | 5 |
| | Cyclohexadimethylsiloxane | 5 | 5 | 5 | 5 |
| | Mixture A | 10 | 0 | 0 | 5 |
| | Mixture B | 0 | 10 | 0 | 5 |

| | | | | |
|---|---|---|---|---|
| Total | 100 | 100 | 100 | 100 |

¹Genapol T080 ® from Clariant
²Hostacerin AMPS from Clariant

Emulsions 1a, 1b, 1c and the mixture 1a+1b are beautiful white creams.

Only emulsions 1a, 1b and 1c display a significant gloss effect.

This is confirmed by the results of the gloss test shown below.

Measurement of gloss of the emulsions in example 1

| | | | | Mixture |
|---|---|---|---|---|
| | Emulsion 1a | Emulsion 1b | Emulsion 1c | 1a + 1b |
| Gloss | 181.7 +/− 0.9 | 177.9 +/− 2.8 | 183.6 +/− 1.5 | 11.3 +/− 2.8 |

### Example 2

### Oil-in-Water Emulsions Comprising an Amphiphilic Polymer Based on Hydroxyethylcellulose

The copolymer used is water-soluble and has a comb structure. It is a cetylhydroxyethylcellulose sold under the designation Natrosol CS Plus 330 by the company Hercules.

The compositions of the emulsions are as follows:

| | | Concentration (wt. %) | | | |
|---|---|---|---|---|---|
| | | Emulsion | Emulsion | Emulsion | Mixture |
| Phase | Name | 2a | 2b | 2c | 2a + 2b |
| A | Distilled water | 77.8 | 77.8 | 87.8 | 77.8 |
| | Preservative | 1 | 1 | 1 | 1 |
| | Cetylhydroxyethylcellulose⁵ | 1 | 1 | 1 | 1 |
| | Polyacrylamidomethyl | 0.2 | 0.2 | 0.2 | 0.2 |
| | propane sulphonic acid | | | | |
| | partially neutralized with | | | | |
| | ammonia and highly crosslinked² | | | | |
| B | Parleam oil | 5 | 5 | 5 | 5 |
| | Cyclohexadimethylsiloxane | 5 | 5 | 5 | 5 |
| | Mixture A | 10 | 0 | 0 | 5 |
| | Mixture B | 0 | 10 | 0 | 5 |

| | | | | |
|---|---|---|---|---|
| Total | 100 | 100 | 100 | 100 |

⁵Natrosol CS Plus 330 ® from the company Hercules
²Hostacerin AMPS ® from Clariant

Measurement of gloss of the emulsions in example 2

| | | | | Mixture |
|---|---|---|---|---|
| | Emulsion 2a | Emulsion 2b | Emulsion 2c | 2a + 2b |
| Gloss | 94.1 +/− 14.9 | 80.1 +/− 6.3 | 169.3 +/− 4.9 | 9.3 +/− 2.4 |

These data show that the mixture of emulsions according to the invention, which corresponds to the application of emulsion 2a and of emulsion 2b from example 2, leads to a film whose gloss after drying is reduced very markedly relative to the films obtained from emulsions 2a and 2b alone or alternatively 2c.

### Example 3

### Oil-in-Water Emulsions Comprising an Amphiphilic Comb Polymer Based on Acrylic Acid

The amphiphilic copolymer used is Viscophobe DB 100®. It is water-soluble and has a comb structure. It is a non-crosslinked copolymer obtained from (meth)acrylic acid, methylacrylate and isocyanate of ethoxylated dimethyl meta-isopropenyl benzyl alcohol.

The compositions of the emulsions are as follows:

| | | Concentration (wt. %) | | | |
|---|---|---|---|---|---|
| | | Emulsion | Emulsion | Emulsion | Mixture 3a + |
| Phase | Name | 3a | 3b | 3c | 3b |
| A | Distilled water | 71.8 | 71.8 | 81.8 | 71.8 |
| | Preservative | 1 | 1 | 1 | 1 |
| | Triethanolamine | 1.2 | 1.2 | 1.2 | 1.2 |
| | Polyacrylate-3 to 25% MA⁷ | 6 | 6 | 6 | 6 |
| B | Parleam oil | 5 | 5 | 5 | 5 |
| | Cyclohexadimethylsiloxane | 5 | 5 | 5 | 5 |
| | Mixture A | 10 | 0 | 0 | 5 |
| | Mixture B | 0 | 10 | 0 | 5 |

| | | | | |
|---|---|---|---|---|
| Total | 100 | 100 | 100 | 100 |

⁷Viscophobe DB 100 ® from Amerchol

Measurement of Gloss of the Emulsions in Example 3

| | | | | Mixture |
|---|---|---|---|---|
| | Emulsion 3a | Emulsion 3b | Emulsion 3c | 3a + 3b |
| Gloss | 163.7 +/− 0.3 | 154 +/− 4.3 | 144.4 +/− 2.9 | 21.1 +/− 7.2 |

These data show that the mixture of emulsions according to the invention, which corresponds to the application of emulsion 3a and of emulsion 3b from example 3, leads to a film whose gloss after drying is reduced very markedly relative to the films obtained from emulsions 3a and 3b alone or alternatively 3c.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic kit that can be used for the care and/or make-up of keratinous substance(s) comprising at least two compositions that are different and are packaged separately with at least one of the compositions being of the emulsion type and comprising at least one amphiphilic polymer, the kit comprising one or more compounds X, one or more compounds Y, and at least one catalyst, with at least one of compounds X and Y being a silicone compound and said compounds X and Y being capable of reacting together by a hydrosilylation reaction when they are brought into contact with one another in the presence of a catalyst, and in which compounds X, Y and the catalyst are not present simultaneously in the same composition.

2. Kit according to claim 1 comprising at least:
i. a first composition containing, in a physiologically acceptable medium, at least one compound X and
ii. a second composition containing, in a physiologically acceptable medium, at least one compound Y,
with at least one of said first and second compositions being in the form of an emulsion and containing at least one amphiphilic polymer, and at least one of said first and second compositions containing in addition at least one catalyst.

3. Kit according to claim 1, wherein said amphiphilic polymer is present in at least one of the first or second compositions at a content between 0.01 and 10% by weight relative to the weight of the composition.

4. Kit according to claim 1, wherein the amphiphilic polymer is water-soluble or water-dispersible.

5. Kit according to claim 4, wherein said water-soluble or water-dispersible polymer is selected from polymers derived from acrylamido-2-methylpropane sulphonic acid (AMPS), polymers derived from acrylic acid, derivatives of polyethers, amphiphilic polymers obtained from natural polymers, and mixtures thereof.

6. Kit according to claim 5, wherein the polymer derived from AMPS is selected from the copolymers of AMPS and of C₁₆-C₁₈ alcohol methacrylate having from 6 to 25 oxyethylenated groups, obtained from methacrylic acid or from a salt of methacrylic acid and of a C₁₆-C₁₈ alcohol oxyethylenated with 6 to 25 moles of ethylene oxide.

7. Kit according to claim 5, wherein the polymer derived from acrylic acid is a non-crosslinked copolymer obtained from (meth)acrylic acid, methylacrylate and dimethyl meta-isopropenyl benzyl isocyanate of ethoxylated alcohol.

8. Kit according to claim 5, wherein the polyether derivative is selected from the polyethylene glycol compounds bearing a C₈-C₂₀ alkyl chain at the ends via a urethane linkage and the block copolymers of ethylene oxide and of propylene oxide, of the following formula:
HO(C₂H₄O)X(C₃H₆O)_{y}(C₂H₄O)_{z}H
in which x, y and z are integers such that x+z is in the range from 2 to 100 and y is in the range from 14 to 60.

9. Kit according to claim 5, wherein the amphiphilic polymer obtained from natural polymers is selected from cellulose derivatives, guar derivatives, starch derivatives and derivatives of acacia gum modified with fatty chains having from 6 to 30 carbon atoms.

10. Kit according to claim 1, wherein the amphiphilic polymer is fat-soluble or fat-dispersible.

11. Kit according to claim 10, wherein the fat-soluble or fat-dispersible polymer is selected from polyolefin derivatives, dimethicone copolyols, crosslinked elastomers of emulsifying organopolysiloxanes, derivatives of polyethers, polyhydroxystearate derivatives and mixtures thereof.

12. Kit according to claim 1, wherein compound X is selected from silicone compounds comprising at least two unsaturated aliphatic groups.

13. Kit according to claim 12, wherein compound X is a polyorganosiloxane comprising a silicone main chain whose unsaturated aliphatic groups are pendant from the main chain (side group) or located at the ends of the main chain of the compound (end group).

14. Kit according to claim 13, wherein compound X bears at least one polar group.

15. Kit according to claim 1, wherein compound X is selected from the polyorganosiloxanes comprising at least two unsaturated aliphatic groups each attached to a silicon atom.

16. Kit according to claim 1, wherein compound X is selected from the polyorganosiloxanes containing siloxane units of formula:
$${R}_{m} {R}^{′} {SiO}_{( \frac{3 - m}{2} )} \qquad ( I )$$
in which:
R represents a linear or cyclic, monovalent hydrocarbon group, having from 1 to 30 carbon atoms,
m is equal to 1 or 2 and
R′ represents:
an unsaturated aliphatic hydrocarbon group having from 2 to 10 carbon atoms or
an unsaturated cyclic hydrocarbon group having from 5 to 8 carbon atoms.

17. Kit according to claim 16, wherein the polyorganosiloxane of formula (I) is such that R′ represents a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms.

18. Kit according to claim 16, wherein R represents an alkyl radical having from 1 to 10 carbon atoms or alternatively a phenyl group, and R′ is a vinyl group.

19. Kit according to claim 13, wherein the polyorganosiloxanes additionally comprise units of formula:
$${R}_{n} {SiO}_{( \frac{4 - n}{2} )} \qquad ( II )$$
in which R represents a linear or cyclic, monovalent hydrocarbon group, having from 1 to 30 carbon atoms, and n is equal to 1, 2 or 3.

20. Kit according to claim 1, wherein compound X is selected from organic oligomers or polymers, hybrid organic/silicone oligomers or polymers, said oligomers or polymers bearing at least 2 unsaturated reactive aliphatic groups.

21. Kit according to claim 1, wherein compound Y comprises at least two free Si—H groups.

22. Kit according to claim 1, wherein compound Y is selected from the polyorganosiloxanes comprising at least one alkylhydrogenosiloxane unit with the following formula:
$${R}_{p} H {SiO}_{( \frac{3 - p}{2} )} \qquad ( III )$$
in which:
R represents a linear or cyclic, monovalent hydrocarbon group, having from 1 to 30 carbon atoms or a phenyl group, and p is equal to 1 or 2.

23. Kit according to claim 22, wherein compound Y is such that the radicals R represent a C₁-C₁₀ alkyl group.

24. Kit according to claim 21, in which Y is a polyorganosiloxane comprising at least two alkylhydrogenosiloxane units of formula —(H₃C)(H)Si—O—.

25. Kit according to claim 1, wherein the catalyst is a catalyst based on platinum or tin.

26. Kit according to claim 25, wherein the catalyst is present at a content ranging from 0.0001 to 20 wt. % relative to the total weight of the composition containing it.

27. Kit according to claim 1, wherein compound X is a polydimethylsiloxane with vinylic end groups and compound Y is a polymethylhydrogenosiloxane.

28. Kit according to claim 1, wherein compound X bears at least one polar group that is able to form a hydrogen bond with keratinous substances.

29. Kit according to claim 1, comprising, in at least one of the compositions, a filler selected from silica or surface-treated silica.

30. Kit according to claim 1, wherein compound X has a weight-average molecular weight (Mw) in the range from 150 to 1000 000.

31. Kit according to claim 1, wherein compound Y has a weight-average molecular weight (Mw) in the range from 200 to 1000 000.

32. Kit according to claim 1, wherein compound X represents from 0.1 to 95 wt. % relative to the total weight of the composition containing it.

33. Kit according to claim 1, wherein compound Y represents from 0.1 to 95 wt. % relative to the total weight of the composition containing it.

34. Kit according to claim 1, wherein compounds X and Y are present in the compositions in a molar ratio X/Y in the range from 0.05 to 20.

35. Kit according to claim 1., wherein the amphiphilic polymer is present in a composition in a weight ratio of amphiphilic polymer to compound X or Y between 0.01 and 1.

36. Kit according to claim 1, wherein the first and second compositions are packaged separately in the same packaging article.

37. Kit according to claim 1, wherein at least one of the first and second compositions comprises a liquid fatty phase.

38. Kit according to claim 37, wherein the liquid fatty phase comprises at least one oil.

39. Kit according to claim 38, wherein the oil or oils are present at a content ranging from 2 to 60 wt. %.

40. Kit according to claim 1, wherein at least one of the first and second compositions comprises an aqueous phase.

41. Kit according to claim 1, wherein the composition or compositions are in the form of an water-in-oil, oil-in-water, water-in-oil-in-water or oil-in-water-in-oil emulsion.

42. Kit according to claim 1, wherein at least one of the compositions comprises at least one colorant.

43. Kit according to claim 1, for use as a composition for covering the skin of the body or of the face.

44. Method of cosmetic care and/or of make-up of keratinous substance(s) comprising at least the application on keratinous substances (a) of at least one amphiphilic polymer, (b) of one or more compounds X, (c) of one or more compounds Y, with at least one of compounds X and Y being a silicone compound and said compounds X and Y being capable of reacting together by a hydrosilylation reaction in the presence of a catalyst, when they are brought into contact with one another, and (d) at least one catalyst, and applications (a), (b), (c) and (d) can be simultaneous or consecutive in any order provided that it promotes the interaction of said compounds X and Y.

45. Method according to claim 44, comprising applying, on said keratinous substances, at least one composition of the emulsion type comprising, in a physiologically acceptable medium, at least one compound X, at least one compound Y, at least one catalyst, and at least one amphiphilic polymer.

46. Method according to claim 45, characterized in that said composition is obtained by mixing, at the time of use, a first composition comprising at least compound X and a second composition comprising at least compound Y, at least one of the first and second compositions being an emulsion and containing at least one amphiphilic polymer, and at least one of the first ans second compositions additionally containing at least one catalyst.

47. Cosmetic method for make-up and/or care of keratinous substances, comprising the application on said keratinous substances:
of at least one layer of a first composition containing, in a physiologically acceptable medium, at least one compound X;
of at least one layer of a second composition containing, in a physiologically acceptable medium, at least one compound Y,
at least one of compounds X and Y being a silicone compound, said compounds X and Y being capable of reacting together by a hydrosilylation reaction in the presence of a catalyst, when they are brought into contact with one another, with at least one of said first and second compositions being an emulsion and comprising at least one amphiphilic polymer, at least one of the first and second compositions additionally containing at least one catalyst.

48. Method according to claim 47, wherein the first and/or second compositions are defined as at least two compositions that are different and are packaged separately with at least one of the compositions being of the emulsion type and comprising at least one amphiphilic polymer, the kit comprising one or more compounds X, one or more compounds Y, and at least one catalyst, with at least one of compounds X and Y being a silicone compound and said compounds X and Y being capable of reacting together by a hydrosilylation reaction when they are brought into contact with one another in the presence of a catalyst, and in which compounds X, Y and the catalyst are not present simultaneously in the same composition.

49. Method according to claim 47, including a supplementary stage comprising depositing, on the layer or layers of compositions comprising X and Y, at least one layer of a third composition comprising a film-forming polymer and an organic or aqueous solvent medium.

50. Cosmetic kit that can be used for the care and/or make-up of keratinous substance(s), comprising at least two compositions that are different and are packaged separately with at least one of the compositions being of the emulsion type and comprising at least one amphiphilic polymer, the kit comprising one or more compounds X, one or more compounds Y, and optionally at least one catalyst, with at least one of compounds X and Y being a silicone compound and said compounds X and Y being capable of reacting together by a condensation reaction when they are brought into contact with one another, and in which the compounds X, Y and the catalyst, when it is present, are not present simultaneously in the same composition.
